(19) United States
(12) Patent Application Publication
(10) Pub. No.: US 2026/0308135 A1
(43) Pub. Date: Oct. 8, 2026

(54) Acetoacetylated Alcohols

(71) Applicant: ARXADA AG, Visp (CH)

(72) Inventors: Ulrich Mayerhoeffer, Visp (CH); Stefan Ellinger, Visp (CH); Eva Kirchner, Visp (CH); Sora Pluess, Visp (CH)

(21) Appl. No.: 19/473,902

(22) PCT Filed: Apr. 9, 2024
(86) PCT No.: PCT/EP2024/059557

§ 371 Date: Oct. 9, 2025

(30) Foreign Application Priority Data
Apr. 11, 2023 (EP) 23167304.7

(51) Int. Cl. C07D 305/06; C08K 5/1525; C09D 7/63; C09J 11/06

(52) U.S. Cl. CPC C07D 305/06; C08K 5/1525; C09D 7/63; C09J 11/06

(57) ABSTRACT

The present invention relates to acetoacetylated alcohols further having a cyclic ether functionality such as epoxy or oxetane, compositions comprising acetoacetylated alcohols further having a cyclic ether functionality, and materials derived from said compositions. The present invention further relates to a process of manufacturing acetoacetylated alcohols.

### TECHNICAL FIELD

The present invention relates to acetoacetylated alcohols further having a cyclic ether functionality such as epoxy or oxetane, compositions comprising acetoacetylated alcohols further having a cyclic ether functionality, and materials derived from said compositions. The present invention further relates to a process of manufacturing acetoacetylated alcohols.

### TECHNOLOGICAL BACKGROUND

Molecules having functionalities such as acetoacetylated alcohols and cyclic ethers are of great interest for 3D-printing, coating, adhesive, composite and construction industry (US 2016/0312042, U.S. Pat. No. 6,214,159). Of particular interest are molecules combining these functionalities. The research on suitable molecules is limited, so far. CN115417835 pertains free radical-cationic hybrid monomer derived from itaconic acid oxetane and a preparation method and application thereof. CN110698437 is directed to a preparation method and applications of acrylic acid(3-ethyl-3-oxetanyl)methyl ester.

Many of the current composite and construction materials based on acetoacetylated alcohols or based on cyclic ethers have limitations or disadvantages that restrict their use as e.g. thermosetting resins in high-temperature applications and use of easy processing.

Lacotte et al. (ChemMedChem, 2013, 8(1), 104-111) and Oku et al. (Journal of Organic Chemistry, 2000, 65(7), 1899-1906) disclose processes towards (3-methyloxetan-3-yl)methyl 3-oxobutanoate, which however merely provide yields of less than 80% and may even need organic stannanes as catalyst.

Against this background, there is a need for specific acetoacetylated alcohols further having a cyclic ether functionality, as well as efficient processes towards respective molecules in order to provide coatings, composite and construction materials having improved properties.

It is an object of the present invention to provide molecules combining the functionalities of acetoacetylated alcohols and cyclic ethers. It is a further object of the present invention to provide compositions suitable for 3D-printing or for the manufacturing of coatings, composite and construction materials. It is a further object of the present invention to provide an efficient, e.g. atom and/or yield efficient, process of manufacturing molecules combining the functionalities of acetoacetylated alcohols and cyclic ethers. It is a further object of the present invention to provide a safe and/or easy to handle process of manufacturing molecules combining the functionalities of acetoacetylated alcohols and cyclic ethers. It is a further object of the present invention to provide coatings, composite and construction materials having improved properties (e.g. regarding flexibility in the field of application, film building, adhesion to substrates such as metal, mineral, and wood surface, resistance to impact, resistance to environmental condition, adhesion and cohesion).

### SUMMARY OF THE INVENTION

In a first aspect, the present invention relates to a compound of formula (1)

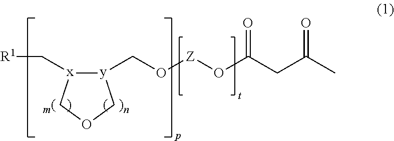

wherein
R¹ is OH, C1-C10-alkyl, C1-C10-haloalkyl, C1-C10-alkoxy, C2-C10-alkenyl, C2-C10-alkynyl, C3-C10-cycloalkyl, C3-C10-cycloalkenyl, C3-C10-aryl, C3-C10-aryloxy, or OR^{A};
Z is within each unit independently C1-C10-alkylene, C2-C10-alkenylene, C2-C10-alkynylene, C3-C10 bivalent aryl, C(O)—C3-C10-aryl-C(O), C1-C10-alkylene-C3-C10-aryl-C1-C10-alkylene, or C3-C10-aryl-C1-C10-alkylene-C3-C10-aryl, wherein each carbon atom of the aforementioned substituents may independently be unsubstituted or substituted with one or more same of different of R^{B};
within each unit independently x and y are either both CH or x is C and y is not present so that the respective unit corresponds to a spiro cyclic moiety of formula (SC)

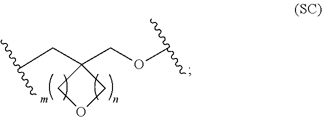

m is independently 0, 1, or 2;
n is independently 0, 1, or 2;
wherein within one unit when y is not present at least one of m or n is 1;
p is an integer of 1 to 6;
R^{A} is a moiety of formula (A-1) or formula (A-2)

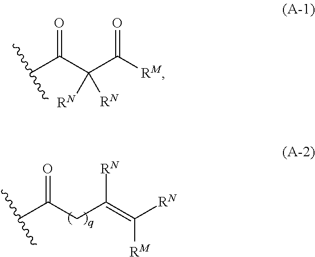

wherein
R^{N} is independently H, halogen, or C1-C3-alkyl;
R^{M} is C1-C6-alkyl or C1-C6-haloalkyl;
R^{B} is halogen, C1-C6-alkyl, or C1-C6-alkoxy;
t is 0 or an integer of 1 to 10000; and
q is 0 or an integer of 1 to 6.

According to a second aspect, the present invention relates to a process of manufacturing an acetoacetylated alcohol, the process comprising the step of reacting an alcohol of formula (20)

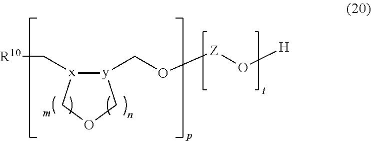

in the presence of a catalyst (C1) with diketene, wherein
R¹⁰ is H, OH, C1-C10-alkyl, C1-C10-haloalkyl, C1-C10-alkoxy, C2-C10-alkenyl, C2-C10-alkynyl, C3-C10-cycloalkyl, C3-C10-cycloalkenyl, C3-C10-aryl, C3-C10-aryloxy, or OR^{A};
Z is within each unit independently C1-C10-alkylene, C2-C10-alkenylene, C2-C10-alkynylene, C3-C10 bivalent aryl, C(O)—C3-C10-aryl-C(O), C1-C10-alkylene-C3-C10-aryl-C1-C10-alkylene, or C3-C10-aryl-C1-C10-alkylene-C3-C10-aryl, wherein each carbon atom of the aforementioned substituents may independently be unsubstituted or substituted with one or more same of different of R^{B};
within each unit independently x and y are either both CH or x is C and y is not present so that the respective unit corresponds to a spiro cyclic moiety of formula (SC)

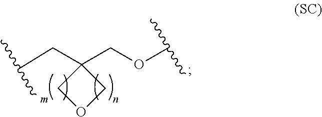

m is independently 0, 1, or 2;
n is independently 0, 1, or 2;
wherein within one unit when y is not present at least one of m or n is 1;
p is an integer of 1 to 6;
R^{A} is a moiety of formula (A-1) or formula (A-2)

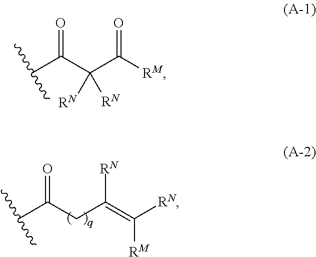

wherein
R^{N} is independently H, halogen, or C1-C3-alkyl;
R^{M} is C1-C6-alkyl or C1-C6-haloalkyl;
R^{B} is halogen, C1-C6-alkyl, or C1-C6-alkoxy;
t is 0 or an integer of 1 to 10000; and
q is 0 or an integer of 1 to 6.

According to a third aspect, the present invention relates to a composition comprising a compound of formula (10) as claimed (and further disclosed in connection with the second aspect) and an additive (A) selected from the group consisting of catalyst (C2), reinforcement fibres, a filler, compounds comprising at least one α,β-unsaturated carbonyl group, epoxies, vinyl compounds, oxetanes, amines, isocyanates, and combinations thereof.

According to a fourth aspect, the present invention relates to a process of manufacturing a 3D-printed object, a coating, a composite or construction material comprising the steps of
i) contacting a composition comprising a compound of formula (10) as defined in in the third aspect with an additive (A) selected from the group consisting of catalyst (C2), reinforcement fibres, a filler, compounds comprising at least one α,β-unsaturated carbonyl group, epoxies, vinyl compounds, oxetanes, amines, isocyanates, and combinations thereof to provide a mixture (M); and
ii) curing said mixture (M).

According to a fifth aspect, the present invention relates to a 3D-printed object, a coating, a composite or construction material obtainable by a process according to the fourth aspect.

According to a sixth aspect, the present invention relates to the use of a compound according to the first aspect or to a composition according to the third aspect in 3D-printing or for producing a coating, an adhesive, a composite or construction material.

According to a seventh aspect, the present invention relates to a method for fastening anchoring elements in boreholes, wherein a composition according to the third aspect is placed in the boreholes and the anchoring element is inserted therein.

According to an eighth aspect, the present invention relates to a 3D printing resin composition comprising a compound of formula (10) as claimed (and further disclosed in connection with the second aspect).

It has surprisingly been found that the inventive compounds and processes solve at least one of the above objects and that the present invention provides inter alia an efficient process of manufacturing molecules combining the functionalities of acetoacetylated alcohols and cyclic ethers, compositions comprising said molecules and/or 3D-prointed objects, adhesives, coatings, composites and construction materials having improved properties.

Without being bound by theory, it is assumed that due to the dual binding functionality (via acetoacetylate (complexation and potential Michael addition) via the cyclic ether (e.g. oxetane) by ring opening polymerization and crosslinking with nucleophiles) the inventive compounds provide high flexibility in the field of application. Thus, the desired properties can be adjusted as necessary, e.g. for improving the film building, for promoting the adhesion to metal, mineral and wood surfaces, for increasing hardness and/or durability, and/or for improved adhesion and/or cohesion. In addition, the inventive compounds may be directly reacted with various functionalities such aldehydes, isocyantes, and azo dye and optionally further crosslinked via Schiffs base reaction when amine cured.

### DETAILED DESCRIPTION OF PREFERRED EMBODIMENTS

In the following, the invention will be explained in more detail.

The term “alkyl” as used herein denotes in each case a straight-chain or branched saturated hydrocarbon group having usually from 1 to 10 carbon atoms, preferably 1 to 6 or 1 to 4 carbon atoms, more preferably 1 to 3 or 1 to 2 or 1 carbon atoms. Examples of an alkyl group are methyl, ethyl, n-propyl, iso-propyl, n-butyl, 2-butyl, iso-butyl, tert-butyl, n-pentyl, 1-methylbutyl, 2-methylbutyl, 3-methyl-butyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethyl-butyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, and the like.

The term “haloalkyl” as used herein denotes in each case a straight-chain or branched saturated hydrocarbon group having usually from 1 to 10 carbon atoms, frequently from 1 to 6 or 1 to 4 carbon atoms, wherein the hydrogen atoms of this group are partially or totally replaced with halogen atoms. Preferred haloalkyl moieties are selected from C₁-C₄-haloalkyl, more preferably from C₁-C₃-haloalkyl or C₁-C₂-haloalkyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “alkenyl” as used herein denotes in each case a straight-chain or branched hydrocarbon group having usually from 2 to 10 carbon atoms, frequently from 2 to 6 or 2 to 4 carbon atoms, with one or more C═C double bonds. The alkenyl moieties, where appropriate, can be of either the (E)- or (Z)-configuration.

The term “alkynyl” as used herein denotes in each case a straight-chain or branched hydrocarbon group having usually from 2 to 10 carbon atoms, frequently from 2 to 8, 2 to 6, or 2 to 4 carbon atoms, with one or more C≡C triple bonds.

The term “alkylene” as used herein refers to a bivalent straight-chain or branched alkyl group, e.g. —(CH2)x- or —CH(CH3)CH2-, wherein x is a positive integer of usually 1 to 20, preferably 1 to 10 or 1 to 5. In the context of the present invention “C1-C10-alkylene” refers to an alkylene moiety with 1, 2, 3, 4, 5, 6, 7, 8, 9, and 10, respectively, carbon atoms, e.g. —CH2— groups; the term “alkylene”, however, not only comprises straight-chain alkylene groups, i.e. “alkylene chains”, but branched alkylene groups, as well. The term “C1-C10-alkylene” refers to an alkylene moiety that is either straight-chain, i.e. an alkylene chain, or branched and has 1, 2, 3, 4, 5, 6, 7, 8, 9, or 10 carbon atoms.

The term “alkenylene” as used herein refers to a bivalent straight-chain or branched alkenyl group as defined herein.

The term “alkynylene” as used herein refers to a bivalent straight-chain or branched alkynyl group as defined herein.

The term “alkoxy” as used herein denotes in each case alkyl or haloalkyl substituents, preferably alkyl substituents, as defined above that are connected to another structural moiety via an oxygen atom (—O—). Exemplary alkoxy groups are methoxy, trifluoromethoxy, ethoxy, 2,2,2-trifluoroethoxy, n-propoxy, iso-propoxy, n-butoxy, sec-butoxy, tert-butoxy, n-pentoxy.

The term “cycloalkyl” refers to a saturated bi- or monocyclic hydrocarbon that has—in general and if not defined otherwise in the specification—a single point of attachment to the remainder of the molecule, with 3, 4, 5, 6, 7, or 8 ring carbon atoms. C3-8-cycloalkyl groups may be unsubstituted or substituted with—unless specified differently elsewhere in this specification—1, 2 or 3 substituents that may be the same of different and are—unless specified differently elsewhere in this specification—selected from the group comprising C1-6-alkyl, C1-6-alkoxy, halogen, hydroxy, unsubstituted or mono- or di-substituted amino. Exemplary C3-8-cycloalkyl groups are cyclopropyl, 2-methyl-cyclopropyl, cyclobutyl, cyclopentyl, cyclohexyl, and cycloheptyl.

The term “cycloalkenyl” as used herein refers to unsaturated (but not aromatic) carbocyclic compounds that can include one or more rings. Thus, the cyclic moiety comprises at least one C═C double bond. In general and if not defined otherwise in the specification—the moiety has a single point of attachment to the remainder of the molecule, with 3, 4, 5, 6, 7, or 8 ring carbon atoms. C3-8-cycloalkenyl groups may be unsubstituted or substituted with—unless specified differently elsewhere in this specification—1, 2 or 3 substituents that may be the same of different and are—unless specified differently elsewhere in this specification—selected from the group comprising C1-6-alkyl, C1-6-alkoxy, halogen, hydroxy, unsubstituted or mono- or di-substituted amino. Examples of “cycloalkenyl” groups include cyclopropenyl, cyclopropadienyl, cyclobutenyl, cyclopentenyl, cyclohexenyl, cyclohexadienyl, cycloheptenyl, cyclooctenyl, norbonenyl, and bicyclo[2.2.2]octenyl.

The term “aryl” (either alone or as part of a larger group, such as e.g. aryloxy) as used herein refers to aromatic ring systems (i.e. fulfilling the Hückel rule—having (4n+π2) electrons, with n being 0 or an integer of preferably 1 to 3) which can be in mono-, bi- or tricyclic form. Examples of such rings include phenyl, naphthyl, or indenyl. Preferred aryl groups are phenyl and naphthyl, phenyl being most preferred. A suitable example for aryloxy rings includes phenoxy. The term “bivalent aryl” as used herein refers to an aryl moiety as defined herein, which has two binding sites to the remainder of the molecule.

The organic moieties mentioned in the above definitions of the variables are—like the term halogen—collective terms for individual listings of the individual group members. The prefix Cn-Cm indicates in each case the possible number of carbon atoms in the group.

The term “C(O)” as used herein denotes a carbonyl moiety (i.e. C═O).

The term “halogen” denotes in each case fluorine, bromine, chlorine, or iodine, in particular chlorine and fluorine.

It is to be understand that if m or n is 0, a direct bond from the respective oxygen to x or y will occur.

It is to be understand that

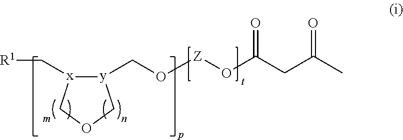

denotes the bond of the respective moiety to the remainder of the molecule.

The terms “about” in the context of the present invention denotes an interval of accuracy that a person skilled in the art will understand to still ensure the technical effect of the feature in question.

The term typically indicates a deviation from the indicated numerical value of ±10%, preferably ±5%, more preferably ±2%, and in particular ±1%.

The term “unit” as used in the context of the present invention denotes the region in between the square brackets, e.g. of formulae (1), (1a), (1b), (1-2a), (1-2b), (1-3a), (1-3b), (10), (10a), (10b), (10-2a), (10-2b), (10-3a), (10-3b), (20), (20b), (20-2a), (20-2b), (20-3a), and (20-3b). The unit may e.g. corresponds to a spiro cyclic moiety of formula (SC) or to a cyclic moiety of formula (C). If p and/or tare more than 1, the units are preferably the same, i.e. repeat units.

It needs to be understood that the term “comprising” is not limiting. For the purposes of the present invention, the term “consisting of” is considered to be a preferred embodiment of the term “comprising of”. If hereinafter a group is defined to comprise at least a certain number of embodiments, this is also meant to encompass a group which preferably consists of these embodiments only.

As outlined above, subject of the present invention is in a first aspect a compound of formula (1)

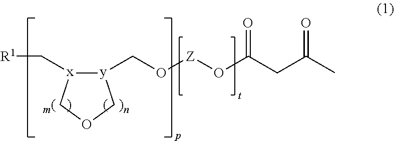

wherein
R¹ is OH, C1-C10-alkyl, C1-C10-haloalkyl, C1-C10-alkoxy, C2-C10-alkenyl, C2-C10-alkynyl, C3-C10-cycloalkyl, C3-C10-cycloalkenyl, C3-C10-aryl, C3-C10-aryloxy, or OR^{A};
Z is within each unit independently C1-C10-alkylene, C2-C10-alkenylene, C2-C10-alkynylene, C3-C10 bivalent aryl, C(O)—C3-C10-aryl-C(O), C1-C10-alkylene-C3-C10-aryl-C1-C10-alkylene, or C3-C10-aryl-C1-C10-alkylene-C3-C10-aryl, wherein each carbon atom of the aforementioned substituents may independently be unsubstituted or substituted with one or more same of different of R^{B};
within each unit independently x and y are either both CH or x is C and y is not present so that the respective unit corresponds to a spiro cyclic moiety of formula (SC)

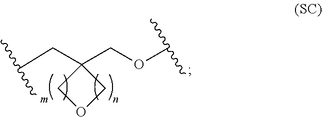

m is independently 0, 1, or 2;
n is independently 0, 1, or 2;
wherein within one unit when y is not present at least one of m or n is 1;
p is an integer of 1 to 6;
R^{A} is a moiety of formula (A-1) or formula (A-2)

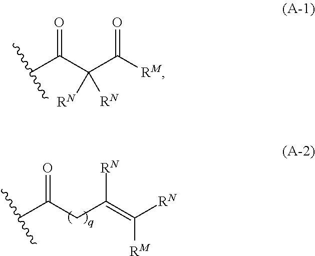

wherein
R^{N} is independently H, halogen, or C1-C3-alkyl;
R^{M} is C1-C6-alkyl or C1-C6-haloalkyl;
R^{B} is halogen, C1-C6-alkyl, or C1-C6-alkoxy;
t is 0 or an integer of 1 to 10000; and
q is 0 or an integer of 1 to 6.

The inventive compounds provide molecules having combined functionalities allowing unique crosslinking properties with two different chemical modes of action and the convenient accessibility of new 3D-Printing, coatings, adhesives, composite or construction materials. The inventive compounds in particular provide the possibility that polymerization of these different functions can be initiated at different stages in the process.

In the following, particular embodiments of the present invention such as moieties are described in further details. It is to be understood that each embodiment is relevant on its own as well as in combination with other embodiments.

The skilled person will understand that when within one unit x and y are both CH the respective unit corresponds to a cyclic moiety of formula (C)

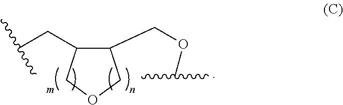

In one embodiment, the compound of formula (1) is a compound of formula (1a)

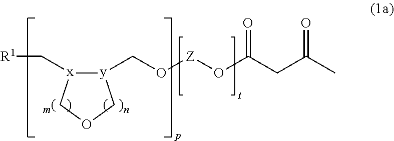

wherein
R¹ is OH, C1-C10-alkyl, C1-C10-haloalkyl, C1-C10-alkoxy, C2-C10-alkenyl, C2-C10-alkynyl, C3-C10-cycloalkyl, C3-C10-cycloalkenyl, C3-C10-aryl, C3-C10-aryloxy, or OR^{A};
Z is within each unit independently C1-C3-alkylene, wherein each carbon atom of the aforementioned substituents may independently be unsubstituted or substituted with one or more same of different of R^{B};
within each unit independently x and y are either both CH or x is C and y is not present so that the respective unit corresponds to a spiro cyclic moiety of formula (SC)

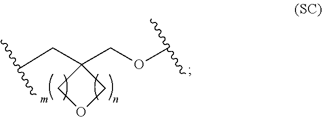

m is independently 0, 1, or 2;
n is independently 0, 1, or 2;
wherein within one unit when y is not present at least one of m or n is 1;
p is an integer of 1 to 6;
R^{A} is a moiety of formula (A-1) or formula (A-2)

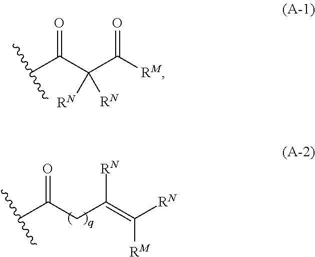

wherein
R^{N} is independently H, halogen, or C1-C3-alkyl;
R^{M} is C1-C6-alkyl or C1-C6-haloalkyl;
R^{B} is halogen or methyl;
t is 0 or an integer of 1 to 2; and
q is 0 or an integer of 1 to 6.

In one embodiment, R¹ is OH, C1-C8-alkyl, C1-C8-haloalkyl, C1-C8-alkoxy, C2-C8-alkenyl, C2-C8-alkynyl, C3-C8-cycloalkyl, C3-C8-cycloalkenyl, C6-C10-aryl, C6-C10-aryloxy, or OR^{A}; preferably OH, C1-C6-alkyl, C1-C6-haloalkyl, C1-C6-alkoxy, C2-C6-alkenyl, C2-C6-alkynyl, C3-C6-cycloalkyl, C3-C6-cycloalkenyl, C6-C10-aryl, C6-C10-aryloxy, or OR^{A}; more preferably OH, C1-C4-alkyl, C1-C4-haloalkyl, C1-C4-alkoxy, C2-C4-alkenyl, C2-C4-alkynyl, C3-C6-cycloalkyl, C3-C6-cycloalkenyl, phenyl, phenoxy, or OR^{A}.

In one embodiment, R¹ is C1-C8-alkyl, C1-C8-haloalkyl, C1-C8-alkoxy, C2-C8-alkenyl, C2-C8-alkynyl, or OR^{A}; preferably C1-C6-alkyl, C1-C6-haloalkyl, C1-C6-alkoxy, C2-C6-alkenyl, C2-C6-alkynyl, or OR^{A}; more preferably C1-C4-alkyl, C1-C4-haloalkyl, C1-C4-alkoxy, C2-C4-alkenyl, C2-C4-alkynyl, or OR^{A}.

In one embodiment, R¹ is C1-C8-alkyl, C1-C8-alkoxy, C2-C8-alkenyl, C2-C8-alkynyl, or OR^{A}; preferably C1-C6-alkyl, C1-C6-alkoxy, C2-C6-alkenyl, C2-C6-alkynyl, or OR^{A}; more preferably C1-C4-alkyl, C1-C4-alkoxy, C2-C4-alkenyl, C2-C4-alkynyl, or OR^{A}.

In a preferred embodiment, R¹ is C1-C6-alkyl or OR^{A}; preferably C1-C4-alkyl or OR^{A}; more preferably C1-C3-alkyl or OR^{A}; and in particular C1-C2-alkyl or OR^{A}.

In one embodiment, R¹ is C1-C8-alkyl, preferably C1-C6-alkyl, more preferably C1-C4-alkyl, even more preferably C1-C3-alkyl, and in particular methyl.

In one embodiment, Z is within each unit independently C1-C8-alkylene, C2-C8-alkenylene, C2-C8-alkynylene, C6-C10 bivalent aryl, C(O)—C6-C10-aryl-C(O), C1-C8-alkylene-C6-C10-aryl-C1-C8-alkylene, or C4-C10-aryl-C1-C8-alkylene-C6-C10-aryl; preferably C1-C6-alkylene, C2-C6-alkenylene, C2-C6-alkynylene, C6-C10 bivalent aryl, C(O)—C6-C10-aryl-C(O), C1-C6-alkylene-C6-C10-aryl-C1-C6-alkylene, or C4-C10-aryl-C1-C6-alkylene-C6-C10-aryl; more preferably C1-C4-alkylene, C2-C4-alkenylene, C2-C4-alkynylene, C6-bivalent aryl, C(O)—C6-aryl-C(O), C1-C4-alkylene-C6-aryl-C1-C4-alkylene, or C4-aryl-C1-C4-alkylene-C6-aryl, wherein each carbon atom of the aforementioned substituents may independently be unsubstituted or substituted with one or more same of different of R^{B}.

In one embodiment, Z is within each unit independently C1-C3-alkylene such as methylene. In one embodiment, the C1-C3-alkylene in unsubstituted.

In one embodiment, Z is within each unit the same.

In one embodiment, R^{B} is halogen, C1-C4-alkyl, or C1-C4-alkoxy; preferably chlorine, fluorine, C1-C2-alkyl, or C1-C2-alkoxy; and in particular chlorine, fluorine, methyl, and methoxy.

In one embodiment, t is 0 or an integer of 1 to 8000; preferably 0 or an integer of 1 to 5000.

In another embodiment, t is 1.

In one embodiment, t is an integer of 1 to 10000 such as 1 to 5000 or 1 to 1000 or 500 to 10000 or 1000 to 10000.

In another embodiment, t is 0. In this connection, the compound of formula (1) or (1a) is a compound of formula (1b)

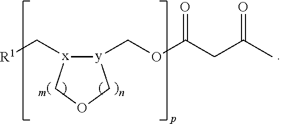

R^{A} is preferably a moiety of formula (A-1) or formula (A-2)

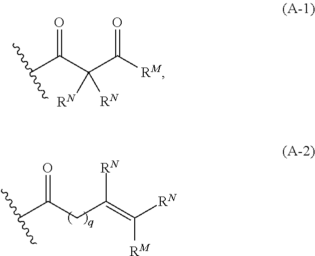

wherein
R^{N} is independently H, halogen, or C1-C2-alkyl, preferably H, fluorine, or methyl, more preferably H or methyl, in particular H;
R^{M} is C1-C5-alkyl or C1-C5-haloalkyl, preferably C1-C4-alkyl or C1-C4-haloalkyl, more preferably C1-C3-alkyl or C1-C3-haloalkyl, in particular C1-C3-alkyl; and
q is 0 or an integer of 1 to 4, preferably 0 or an integer of 1 to 3, more preferably 0 or an integer of 1 to 2, in particular 0 or 1.

In one embodiment,
R¹ is C1-C3-alkyl or OR^{A};
p is 1 or 2;
R^{A} is a moiety of formula (A-1) or formula (A-2)

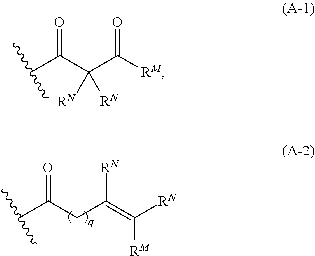

wherein
R^{N} is independently H or methyl, preferably H;
R^{M} is C1-C3-alkyl, preferably methyl; and
q is 0 or an integer of 1 to 3, preferably 0,
preferably wherein each x is C and each y is not present.

In one embodiment, R¹ is OR^{A}, wherein R^{A} is a moiety of formula (A-1)

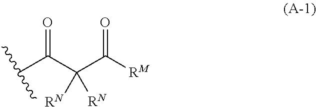

wherein
R^{N} is independently H, halogen, or C1-C3-alkyl, preferably H, fluorine, or methyl, more preferably H or methyl, in particular H;
R^{M} is C1-C6-alkyl or C1-C6-haloalkyl, preferably C1-C4-alkyl or C1-C4-haloalkyl, more preferably C1-C3-alkyl or C1-C3-haloalkyl, in particular C1-C3-alkyl.

In one embodiment, R¹ is OR^{A}, wherein R^{A} is a moiety of formula (A-2)

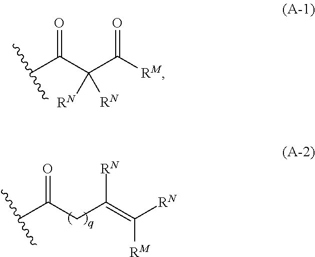

wherein
R^{N} is independently H, halogen, or C1-C3-alkyl, preferably H, fluorine, or methyl, more preferably H or methyl, in particular H;
R^{M} is C1-C6-alkyl or C1-C6-haloalkyl, preferably C1-C4-alkyl or C1-C4-haloalkyl, more preferably C1-C3-alkyl or C1-C3-haloalkyl, in particular C1-C3-alkyl; and
q is 0 or an integer of 1 to 4, preferably 0 or an integer of 1 to 2, more preferably 0 or 1, in particular 0.

In one embodiment, within at least one unit x is C and y is not present.

In one embodiment, within at least one unit x and y are both CH.

In one embodiment, each x is C and each y is not present so that the compound of formula (1) or (1a) is a compound of formula (1-2a)

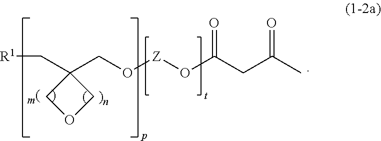

In one embodiment, each x is C and each y is not present and t is 0 so that the compound of formula (1) or (1a) is a compound of formula (1-2b)

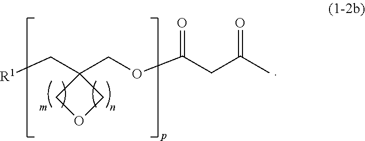

In one embodiment, each x and y are CH so that the compound of formula (1) or (1a) is a compound of formula (1-3a)

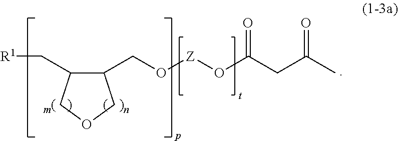

In one embodiment, each x and y are CH and t is 0 so that the compound of formula (1) or (1a) is a compound of formula (1-3b)

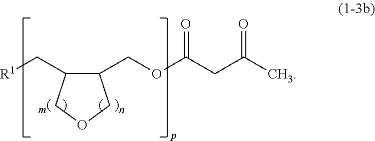

In one embodiment, m is independently 0 or 1, wherein preferably each unit has the same m value.

In one embodiment, n is independently 0 or 1, wherein preferably each unit has the same n value.

In one embodiment, at least one of m or n are independently 0 or 1, preferably each m and n are independently 0 or 1, more preferably each unit has the same m and n value.

In one embodiment, within at least one unit x and y are both CH and m and n are both 0, preferably within each unit x and y are both CH and m and n are both 0.

In one embodiment, within at least one unit x is C and y is not present and m and n are independently 0 or 1 (wherein at least one of m or n is 1), preferably within each unit x is C and y is not present and m and n are independently 0 or 1 (wherein at least one of m or n is 1).

In one embodiment, within at least one unit x and y are CH and m and n are independently 0 or 1, wherein at least one of m or n is 0, preferably within each unit x and y are CH and m and n are independently 0 or 1, wherein at least one of m or n is 0.

In one embodiment, within at least one unit x is C and y is not present and m and n are 1, preferably within each unit x is C and y is not present and m and n are 1.

In one embodiment, p is an integer of 1 to 5, preferably of 1 to 4, more preferably of 1 to 3, and in particular of 1 or 2.

When p is 2 to 6, the respective units are preferably the same (regarding x, y, m, and n), i.e. the units are repeat units.

In one embodiment,
R¹ is C1-C6-alkyl, preferably C1-C3-alkyl, more preferably methyl;
x is C and y is not present; and
p is 1,
preferably wherein m and n are 1.

In one embodiment,
R¹ is C1-C6-alkyl, preferably C1-C3-alkyl, more preferably methyl;
x is C and y is not present;
t is 0 or an integer of 1 to 10000, preferably 0 or 1, more preferably 0; and
p is 1,
preferably wherein m and n are 1.

In one embodiment,
R¹ is C1-C6-alkyl, preferably C1-C3-alkyl, more preferably methyl;
x is C and y is not present;
t is 0 or 1, preferably 0; and
p is 1,
preferably wherein m and n are 1.

In one embodiment,
R¹ is OR^{A};
p is 1;
R^{A} is a moiety of formula (A-1) or formula (A-2)

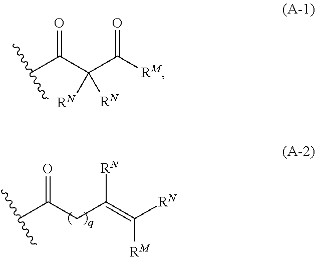

wherein
R^{N} is independently H or methyl, preferably H;
R^{M} is 1-C3-alkyl, preferably methyl; and
q is 0 or an integer of 1 to 3, preferably 0.

In one embodiment,
R¹ is C1-C3-alkyl or OR^{A};
p is 2;
each x is C and each y is not present;
R^{A} is a moiety of formula (A-1) or formula (A-2)

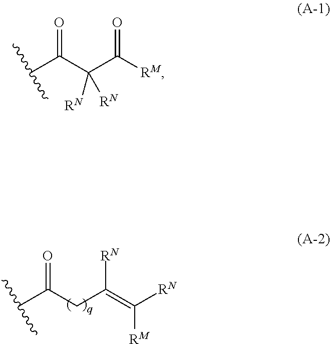

wherein
R^{N} is independently H or methyl, preferably H;
R^{M} is C1-C3-alkyl, preferably methyl; and
q is 0 or an integer of 1 to 3, preferably 0.

In one embodiment, the compound of formula (1) is selected from the group consisting of

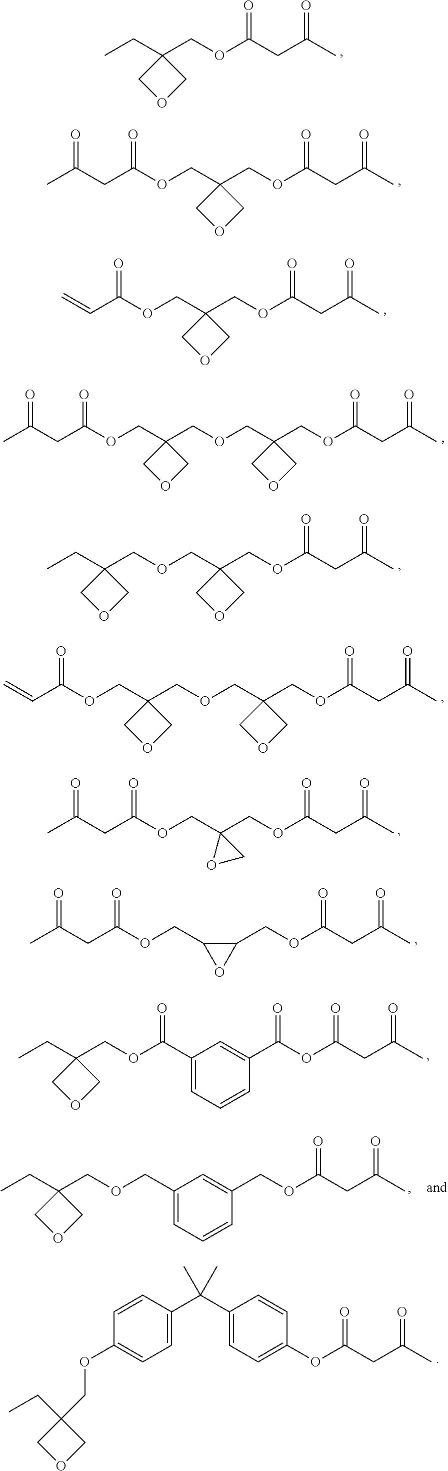

In a preferred embodiment, the compound of formula (1) or (1a) is selected from the group consisting of

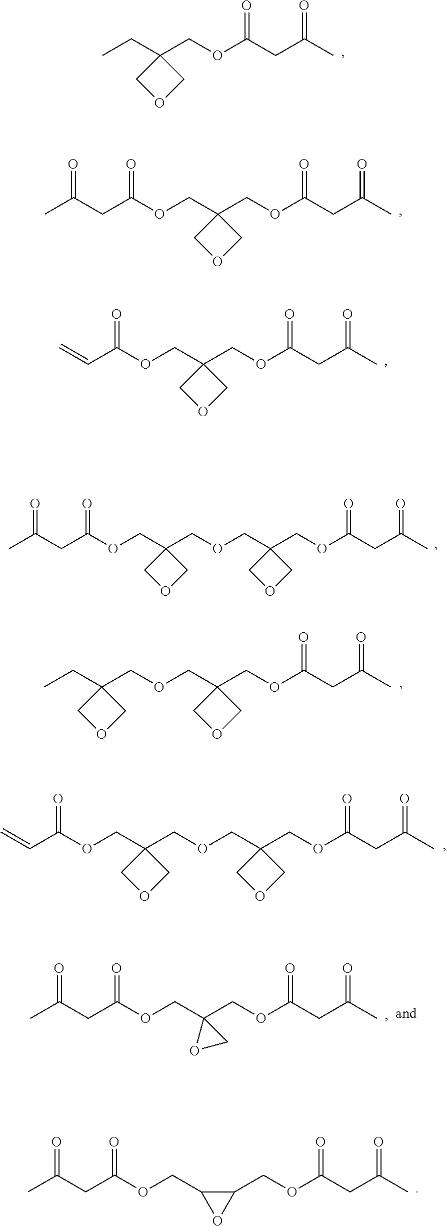

As aforementioned, the present invention further relates in a second aspect to a process of manufacturing an acetoacetylated alcohol, the process comprising the step of reacting an alcohol of formula (20)

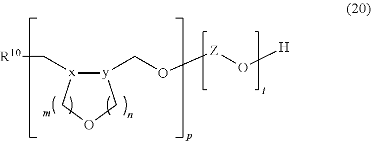

in the presence of a catalyst (C1) with diketene, wherein
R c is H, OH, C1-C10-alkyl, C1-C10-haloalkyl, C1-C10-alkoxy, C2-C10-alkenyl, C2-C10-alkynyl, C3-C10-cycloalkyl, C3-C10-cycloalkenyl, C3-C10-aryl, C3-C10-aryloxy, or OR^{A};
Z is within each unit independently C1-C10-alkylene, C2-C10-alkenylene, C2-C10-alkynylene, C3-C10 bivalent aryl, C(O)—C3-C10-aryl-C(O), C1-C10-alkylene-C3-C10-aryl-C1-C10-alkylene, or C3-C10-aryl-C1-C10-alkylene-C3-C10-aryl, wherein each carbon atom of the aforementioned substituents may independently be unsubstituted or substituted with one or more same of different of R^{B};
within each unit independently x and y are either both CH or x is C and y is not present so that the respective unit corresponds to a spiro cyclic moiety of formula (SC)

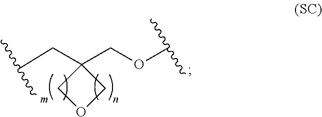

m is independently 0, 1, or 2;
n is independently 0, 1, or 2;
wherein within one unit when y is not present at least one of m or n is 1;
p is an integer of 1 to 6;
R^{A} is a moiety of formula (A-1) or formula (A-2)

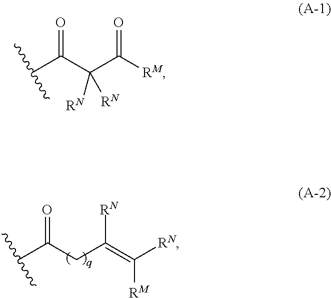

wherein
R^{N} is independently H, halogen, or C1-C3-alkyl;
R^{M} is C1-C6-alkyl or C1-C6-haloalkyl;
R^{B} is halogen, C1-C6-alkyl, or C1-C6-alkoxy;
t is 0 or an integer of 1 to 10000; and
q is 0 or an integer of 1 to 6.

The inventive process provides an efficient synthetic access to acetoacetylated alcohol having additional functionalities. Notable, the inventive process provides the acetoacetylated alcohol in high yield, e.g. up to about quantitative yields. Further, the inventive process provides an economically and environmentally friendly process due to the high atom efficiency, wherein depending on the applied catalyst (C1) said catalyst (C1) can be recycled. Thus, the present invention provides a process having reduced waste. In addition, the inventive process can be run solvent free thus providing high atom efficiency. Finally, the inventive process provides the acetoacetylated alcohol under safe conditions since catalysts such as stannanes containing catalysts can be avoided, which is of particular importance for industrial scale.

Particular embodiments (e.g. regarding x, y, Z, m, n, R^{A}, R^{B}, p, and t) are already above-outlined in connection with the inventive compounds and shall hold for the process, as well. In the following, particular embodiments of the process are described in further detail. It is to be understood that each embodiment is relevant on its own as well as in combination with other embodiments.

In one embodiment, R¹⁰ is H, OH, C1-C8-alkyl, C1-C8-haloalkyl, C1-C8-alkoxy, C2-C8-alkenyl, C2-C8-alkynyl, C3-C8-cycloalkyl, C3-C8-cycloalkenyl, C6-C10-aryl, C6-C10-aryloxy, or OR^{A}; preferably OH, C1-C6-alkyl, C1-C6-haloalkyl, C1-C6-alkoxy, C2-C6-alkenyl, C2-C6-alkynyl, C3-C6-cycloalkyl, C3-C6-cycloalkenyl, C6-C10-aryl, C6-C10-aryloxy, or OR^{A}; more preferably OH, C1-C4-alkyl, C1-C4-haloalkyl, C1-C4-alkoxy, C2-C4-alkenyl, C2-C4-alkynyl, C3-C6-cycloalkyl, C3-C6-cycloalkenyl, phenyl, phenoxy, or OR^{A}.

In one embodiment, R¹⁰ is H, OH, C1-C8-alkyl, C1-C8-haloalkyl, C1-C8-alkoxy, C2-C8-alkenyl, C2-C8-alkynyl, or OR^{A}; preferably H, OH, C1-C6-alkyl, C1-C6-haloalkyl, C1-C6-alkoxy, C2-C6-alkenyl, C2-C6-alkynyl, or OR^{A}; more preferably H, OH, C1-C4-alkyl, C1-C4-haloalkyl, C1-C4-alkoxy, C2-C4-alkenyl, C2-C4-alkynyl, or OR^{A}.

In one embodiment, R¹⁰ is H, OH, C1-C8-alkyl, C1-C8-alkoxy, C2-C8-alkenyl, C2-C8-alkynyl, or OR^{A}; preferably H, OH, C1-C6-alkyl, C1-C6-alkoxy, C2-C6-alkenyl, C2-C6-alkynyl, or OR^{A}; more preferably H, OH, C1-C4-alkyl, C1-C4-alkoxy, C2-C4-alkenyl, C2-C4-alkynyl, or OR^{A}.

In a preferred embodiment, R¹⁰ is H, OH, C1-C6-alkyl, or OR^{A}; preferably is H, OH, C1-C5-alkyl, or OR^{A}; more preferably is H, OH, C1-C4-alkyl, or OR^{A}; even more preferably is H, OH, C1-C3-alkyl, or OR^{A}; and in particular H, OH, C1-C2-alkyl or OR^{A}.

In one embodiment, R¹⁰ is H.

In one embodiment, R¹⁰ is H or C1-C8-alkyl; preferably H or C1-C6-alkyl; more preferably H or C1-C4-alkyl; even more preferably H or C1-C3-alkyl; and in particular H or methyl.

In one embodiment, Z is within each unit independently C1-C8-alkylene, C2-C8-alkenylene, C2-C8-alkynylene, C6-C10 bivalent aryl, C(O)—C6-C10-aryl-C(O), C1-C8-alkylene-C6-C10-aryl-C1-C8-alkylene, or C4-C10-aryl-C1-C8-alkylene-C6-C10-aryl; preferably C1-C6-alkylene, C2-C6-alkenylene, C2-C6-alkynylene, C6-C10 bivalent aryl, C(O)—C6-C10-aryl-C(O), C1-C6-alkylene-C6-C10-aryl-C1-C6-alkylene, or C4-C10-aryl-C1-C6-alkylene-C6-C10-aryl; more preferably C1-C4-alkylene, C2-C4-alkenylene, C2-C4-alkynylene, C6-bivalent aryl, C(O)—C6-aryl-C(O), C1-C4-alkylene-C6-aryl-C1-C4-alkylene, or C4-aryl-C1-C4-alkylene-C6-aryl, wherein each carbon atom of the aforementioned substituents may independently be unsubstituted or substituted with one or more same of different of R^{B}.

In one embodiment, Z is within each unit independently C1-C3-alkylene such as methylene. In one embodiment, the C1-C3-alkylene in unsubstituted.

In one embodiment, within each unit Z is the same.

In one embodiment, R^{B} is halogen, C1-C4-alkyl, or C1-C4-alkoxy; preferably chlorine, fluorine, C1-C2-alkyl, or C1-C2-alkoxy; and in particular chlorine, fluorine, methyl, and methoxy.

In one embodiment, t is 0 or an integer of 1 to 8000, preferably 0 or an integer of 1 to 5000.

In another embodiment, t is 1.

In one embodiment, t is an integer of 1 to 10000 such as 1 to 5000 or 1 to 1000 or 500 to 10000 or 1000 to 10000.

In another embodiment, t is 0. In this connection, the compound of formula (20) is a compound of formula (20b)

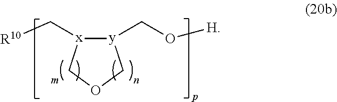

In one embodiment, each x is C and each y is not present so that the alcohol of formula (20) is an alcohol of formula (20-2a)

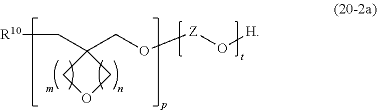

In one embodiment, each x is C and each y is not present and t is 0 so that the alcohol of formula (20) is an alcohol of formula (20-2b)

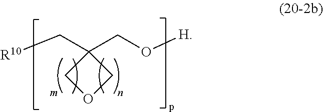

In one embodiment, each x and y are CH so that the alcohol of formula (20) is an alcohol of formula (20-3a)

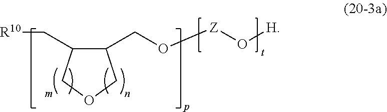

In one embodiment, each x and y are CH and t is 0 so that the alcohol of formula (20) is an alcohol of formula (20-3b)

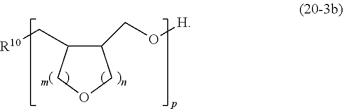

In one embodiment, the acetoacetylated alcohol is a compound of formula (10)

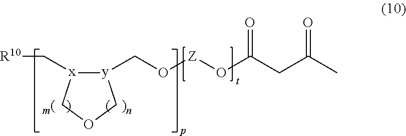

wherein
R¹⁰ is H, OH, C1-C10-alkyl, C1-C10-haloalkyl, C1-C10-alkoxy, C2-C10-alkenyl, C2-C10-alkynyl, C3-C10-cycloalkyl, C3-C10-cycloalkenyl, C3-C10-aryl, C3-C10-aryloxy, or OR^{A};
Z is within each unit independently C1-C10-alkylene, C2-C10-alkenylene, C2-C10-alkynylene, C3-C10 bivalent aryl, C(O)—C3-C10-aryl-C(O), C1-C10-alkylene-C3-C10-aryl-C1-C10-alkylene, or C3-C10-aryl-C1-C10-alkylene-C3-C10-aryl, wherein each carbon atom of the aforementioned substituents may independently be unsubstituted or substituted with one or more same of different of R^{B};
within each unit independently x and y are either both CH or x is C and y is not present so that the respective unit corresponds to a spiro cyclic moiety of formula (SC)

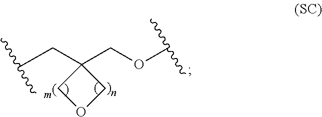

m is independently 0, 1, or 2;
n is independently 0, 1, or 2;
wherein within one unit when y is not present at least one of m or n is 1;
p is an integer of 1 to 6;
R^{A} is a moiety of formula (A-1) or formula (A-2)

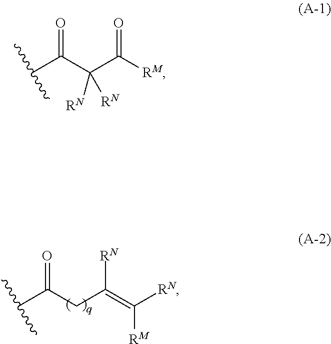

wherein
R^{N} is independently H, halogen, or C1-C3-alkyl;
R^{M} is C1-C6-alkyl or C1-C6-haloalkyl;
R^{B} is halogen, C1-C6-alkyl, or C1-C6-alkoxy;
t is 0 or an integer of 1 to 10000; and
q is 0 or an integer of 1 to 6.

In one embodiment, Z is within each unit independently C1-C3-alkylene, R^{B} is halogen or methyl, and t is 0 or an integer of 1 to 2. In this connection, it is referred to a compound of formula (10a). In one embodiment, the C1-C3-alkylene in unsubstituted.

In one embodiment, t is 0 so that the compound of formula (10) or (10a) is a compound of formula (10b)

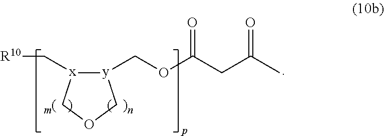

In one embodiment, each x is C and each y is not present so that the compound of formula (10) or (10a) is a compound of formula (10-2a)

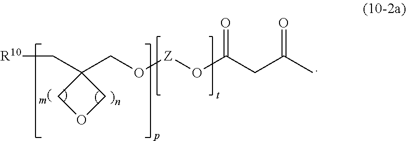

In one embodiment, each x is C and each y is not present and t is 0 so that the compound of formula (10) or (10a) is a compound of formula (10-2b)

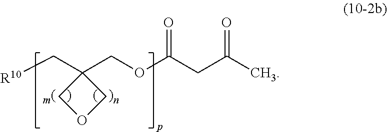

In one embodiment, each x and y are CH so that the compound of formula (10) or (10a) is a compound of formula (10-3a)

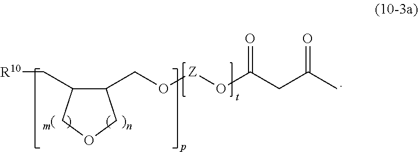

In one embodiment, each x and y are CH and t is 0 so that the compound of formula (10) or (10a) is a compound of formula (10-3b)

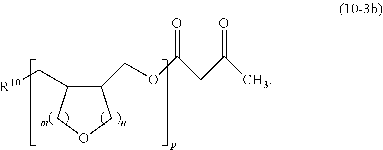

In one embodiment, the acetoacetylated alcohol is a compound of formula (1) or (1a) according to the first aspect (including all embodiments).

In one embodiment, the catalyst (C1) is a tertiary amine, preferably selected from the group consisting of trialkyl amine such as trimethylamine and triethylamine (TEA), 1,4-diazabicyclo[2.2.2]octane (DABCO), N,N-dimethylpyridin-4-amin, 1,1,3,3-tetramethylguanidine (TMG), N,N-dimethylisopropylamine, 1,8-diazabicyclo(5.4.0)undec-7-ene (DBU), 1,5-diazabicyclo(4.3.0)non-5-ene (DBN), resin bond teriary amines, and combinations thereof, and/or carboxylic acid salts, preferably selected form the group consisting of sodium acetate, potassium acetate, and combinations thereof.

In one embodiment the catalyst (C1) is a tertiary amine selected from the group consisting of trimethylamine, trimethylamine, and combinations thereof. In this connection, the catalyst may e.g. be recycled via distillation.

In one embodiment, the catalyst (C1) is 1,4-diazabicyclo[2.2.2]octane (DABCO). In this connection, the catalyst may e.g. be recycled vis lonexchanger or filtered off as polymerbond DABCO resin.

In one embodiment, the diketene and the alcohol of formula (20) are present in the reaction in a molar ratio of about 1:2 to about 10:1, preferably of about 1:1.5 to about 6:1, more preferably of about 1:1.3 to about 3:1, even more preferably of about 1:1.1 to about 2.1:1, and in particular of about 1:1 to about 2:1. In a specific embodiment, the diketene and the alcohol of formula (20) are present in the reaction in a molar ratio of about 1:4 to about 4:1, preferably of about 1:2 to about 2:1, more preferably of about 1:1.5 to about 1.5:1, even more preferably of about 1:1.1 to about 1.1:1, and in particular of about 1:1. In another specific embodiment, the diketene and the alcohol of formula (20) are present in the reaction in a molar ratio of about 1:2 to about 10:1, preferably of about 1:1 to about 6:1, more preferably of about 1.5:1 to about 4:1, even more preferably of about 1.8:1 to about 2.2:1, and in particular of about 2:1.

In one embodiment, the diketene is present in the reaction from about 1 to about 1.3 eq, preferably from about 1 to about 1.2 eq, more preferably from about 1 to about 1.1 eq, even more preferably from about 1 to about 1.05 eq, of the total molar equivalents of hydroxyl residues in alcohol of formula (20).

In one embodiment, the reaction further comprises a solvent, preferably a polar aprotic solvents such as, acetone, ethyl acetate, and/or butyl acetate.

In a preferred embodiment, the reaction is performed in the absence of a solvent.

In one embodiment, the catalyst (C1) is present in the reaction from about 0.0001 to about 0.1 eq, more preferably from about 0.0002 to about 0.01 eq, even more preferably from about 0.0003 to about 0.005, and in particular from about 0.0004 to about 0.0025 eq, of the molar amount of the alcohol of formula (20).

In one embodiment, the alcohol of formula (20) is precharged to a suitable container (e.g. vessel, stirred reactor and the like), and more preferably the catalyst (C1) is added thereto.

In one embodiment, the diketene is charged, preferably dosed stepwise, to a mixture of the alcohol of formula (20) and the catalyst (C1). Addition of the diketene is preferably performed under temperature control.

In general, the reaction time of said reaction is from about 1 minute to about 15 hours, preferably form about 5 minutes to about 12 hours.

In one embodiment, the reaction is complete after charging of the diketene and stirring is stopped after charging.

If the alcohol of formula (20) is solid at ambient temperature, the reaction temperature of said reaction is preferably above the melting point of said alcohol of formula (20).

In one embodiment, the reaction is done at a temperature from about 0 to about 150° C., preferably from about 5 to about 120° C., more preferably from about 20 to about 80° C., and in particular from about 25 to about 50° C.

In one embodiment, the charging is done at a temperature of from about 0 to about 150° C., preferably of from about 5 to about 120° C., more preferably of from about 20 to about 80° C., and in particular of from about 25 to about 50° C.

Preferably, the reaction is done under atmospheric pressure. The reaction can however also be done under a pressure above atmospheric pressure, for example when a reaction temperature is chosen above the boiling point of any of the components of the reaction mixture.

After the reaction, the acetoacetylated alcohol can be isolated by standard means known to the skilled person, by e.g. concentration by vacuum distillation if the reaction is performed in a solvent or simple decharging from the reaction container.

In one embodiment, the process is conducted under inert gas atmosphere (e.g. nitrogen or argon).

In one embodiment, the process further comprises an upstream step of synthesizing the compound of formula (20). In particular, if t is an integer of 1 to 10000, the process may comprise an upstream step. Any suitable reaction may be possible in order to obtain the respective ether/ester compound, which is not yet acetylated. One suitable starting material may e.g. be a compound of formula (20b).

Since the compound of formula (20) is in general stable, an upstream synthesis prior the inventive reaction is not necessary.

As aforementioned, the present invention further relates in a third aspect to a composition comprising a compound of formula (10)

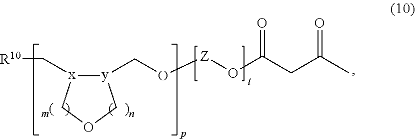

wherein
R¹⁰ is H, OH, C1-C10-alkyl, C1-C10-haloalkyl, C1-C10-alkoxy, C2-C10-alkenyl, C2-C10-alkynyl, C3-C10-cycloalkyl, C3-C10-cycloalkenyl, C3-C10-aryl, C3-C10-aryloxy, or OR^{A};
Z is within each unit independently C1-C10-alkylene, C2-C10-alkenylene, C2-C10-alkynylene, C3-C10 bivalent aryl, C(O)—C3-C10-aryl-C(O), C1-C10-alkylene-C3-C10-aryl-C1-C10-alkylene, or C3-C10-aryl-C1-C10-alkylene-C3-C10-aryl, wherein each carbon atom of the aforementioned substituents may independently be unsubstituted or substituted with one or more same of different of R^{B};
within each unit independently x and y are either both CH or x is C and y is not present so that the respective unit corresponds to a spiro cyclic moiety of formula (SC)

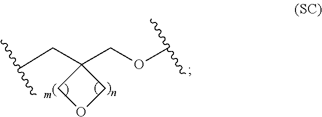

m is independently 0, 1, or 2;
n is independently 0, 1, or 2;
wherein within one unit when y is not present at least one of m or n is 1;
p is an integer of 1 to 6;
R^{A} is a moiety of formula (A-1) or formula (A-2)

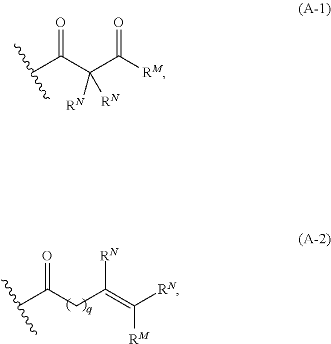

wherein
R^{N} is independently H, halogen, or C1-C3-alkyl;
R^{M} is C1-C6-alkyl or C1-C6-haloalkyl;
R^{B} is halogen, C1-C6-alkyl, or C1-C6-alkoxy;
t is 0 or an integer of 1 to 10000; and
q is 0 or an integer of 1 to 6,
and an additive (A) selected from the group consisting of catalyst (C2), reinforcement fibres, a filler, compounds comprising at least one α,β-unsaturated carbonyl group, epoxies, vinyl compounds, oxetanes, amines, isocyanates, and combinations thereof.

Particular embodiments (e.g. regarding x, y, Z, m, n, R¹, R¹⁰, R^{A}, R^{B}, p, and t) are already above-outlined in connection with the inventive compounds and the inventive process and shall hold for the composition, as well. In the following, particular embodiments of the composition are described in further detail. It is to be understood that each embodiment is relevant on its own as well as in combination with other embodiments.

It is to be understood that the additive (A) (e.g. compounds comprising at least one α,β-unsaturated carbonyl group, epoxies or oxetane) is structurally different to the compound of formula (10).

Preferably, the composition is a 3D-printing, a coating, an adhesive, a composite or a construction composition. The term “coating” as used herein encompasses paints.

In one embodiment, the compound of formula (10) is a compound of formula (10a)

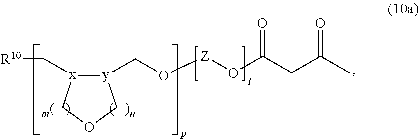

wherein
R¹⁰ is H, OH, C1-C10-alkyl, C1-C10-haloalkyl, C1-C10-alkoxy, C2-C10-alkenyl, C2-C10-alkynyl, C3-C10-cycloalkyl, C3-C10-cycloalkenyl, C3-C10-aryl, C3-C10-aryloxy, or OR^{A};
Z is within each unit independently C1-C3-alkylene, wherein each carbon atom of the aforementioned substituents may independently be unsubstituted or substituted with one or more same of different of R^{B};
within each unit independently x and y are either both CH or x is C and y is not present so that the respective unit corresponds to a spiro cyclic moiety of formula (SC)

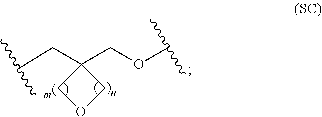

m is independently 0, 1, or 2;
n is independently 0, 1, or 2;
wherein within one unit when y is not present at least one of m or n is 1;
p is an integer of 1 to 6;
R^{A} is a moiety of formula (A-1) or formula (A-2)

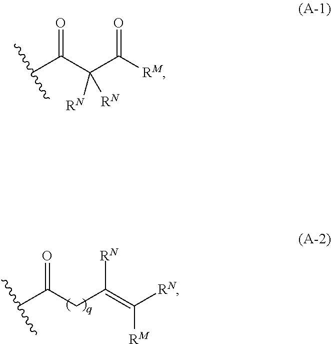

wherein
R^{N} is independently H, halogen, or C1-C3-alkyl;
R^{M} is C1-C6-alkyl or C1-C6-haloalkyl;
R^{B} is halogen or methyl;
t is 0 or an integer of 1 to 2; and
q is 0 or an integer of 1 to 6,
and an additive (A) selected from the group consisting of catalyst (C2), reinforcement fibres, a filler, compounds comprising at least one α,β-unsaturated carbonyl group, epoxies, vinyl compounds, oxetanes, amines, isocyanates, and combinations thereof.

In one embodiment, the catalyst (C2) is selected from the group consisting of aliphatic mono-, di- and polyamines; aromatic mono-, di- and polyamines; carbocyclic mono-, di and polyamines; heterocyclic mono-, di- and polyamines; compounds containing a five- or six-membered nitrogen-containing heterocyclic ring; hydroxyamines; phosphines; phenols; and mixtures thereof. Preferably, the catalyst (C2) is a tertiary amine, more preferably selected form the group consisting of trialkyl amine such as triethylamine (TEA), 1,4-diazabicyclo[2.2.2]octane (DABCO), N,N-dimethylpyridin-4-amin, 1,1,3,3-tetramethylguanidine (TMG), N,N-dimethylisopropylamine, 1,8-diazabicyclo(5.4.0)undec-7-ene (DBU), and 1,5-diazabicyclo(4.3.0)non-5-ene (DBN), and in particular selected from the group consisting of 1,1,3,3-tetramethylguanidine (TMG), 1,4-diazabicyclo[2.2.2]octane (DABCO), triethylamine (TEA), N,N-dimethylisopropylamine, 1,8-diazabicyclo(5.4.0)undec-7-ene (DBU), and 1,5-diazabicyclo(4.3.0)non-5-ene (DBN).

In one embodiment, the catalyst (C2) is a cationic catalyst such as iodonium and sulfonium salts. Suitable catalysts include but are not limited to diaryliodonium compounds or triarylsulfonium compounds paired with anions such as BF4-, B(C6F5)4-, PF6-, AsF6-, SbF6-, CF3SO3-, C4F9SO3-,

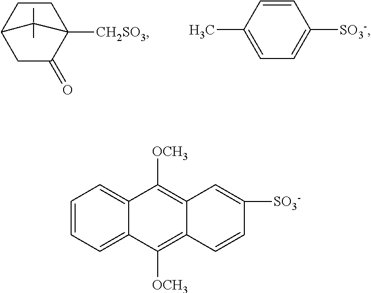

and variations thereof. Suitable iodonium salts may be selected from the compounds selected from the group consisting of

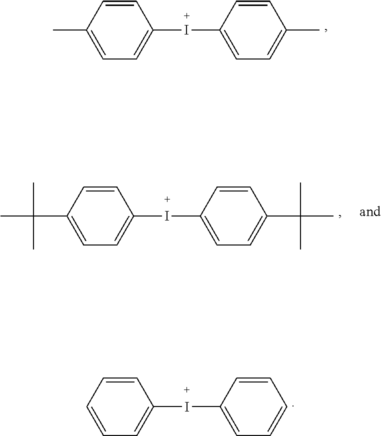

Suitable sulfonium salts may be selected from the group consisting of

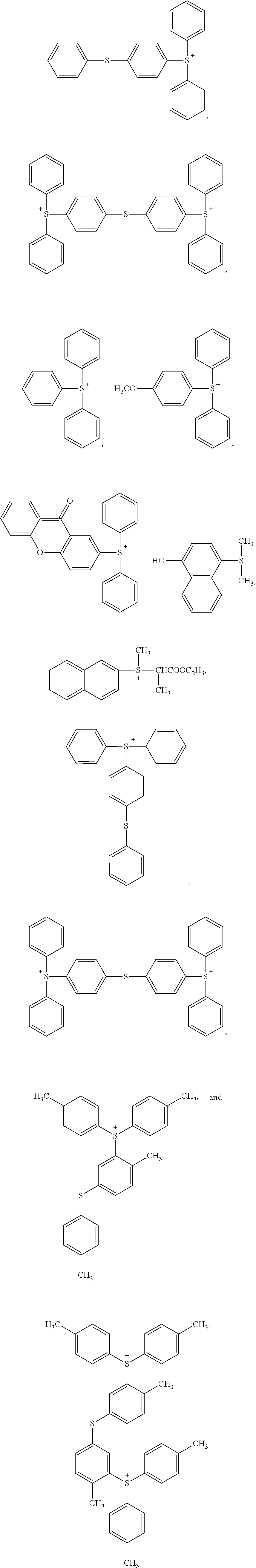

Examples of such catalyst are bis-(4-dodecylphenyl)iodonium hexafluroantimonate in glycidyl ether, (sulfanediyldibenzene-4,1-diyl)bis(diphenylsulfonium) bis(hexafluoroantimonate) in digycidyl ether, and bis-(4-t-butylphenyl)-iodonium hexafluorophosphate. Further suitable cationic catalysts are available from Arkema.

In one embodiment, the catalyst (C2) is a radical catalyst. Suitable radical catalysts include but are not limited to dialkyl peroxides, diacyl peroxides, and azo compounds. Particularly suitable radical catalysts include dicumyl peroxide and 2,5-Dimethyl-2,5-di-(tert-butylperoxy)hexyne-3 (Trigonox 145-E85).

The amount of the catalyst (C2) can be varied to adapt to different applications and needs. Typically, the amount of the catalyst (C2) ranges from 0.05 to 10.0 wt %, more preferably from 0.1 to 7 wt %, even more preferably from 0.15 to 5 wt % based on the total amount of compound of formula (10) or (10a).

In one embodiment, the reinforcement fibres are selected from the group consisting of carbon fibres; glass fibres, preferably E glass fibres or S glass fibres; aramid fibres (including KEVLAR®); basalt fibres (geotextile fibers); natural fibres, preferably flax, hemp, jute or sisal; fleeces; and woven fabrics (multi-layered or single layered); and mixtures thereof.

In a preferred embodiment, the reinforcement fibres are carbon fibres such as polyacrylonitrile PAN based carbon fibres, glass fibres, basalt fibres, aramid fibres or natural fibres, or mixtures thereof.

In another preferred embodiment the reinforcement fibres are glass fibres, carbon fibres or aramid fibres, or mixtures thereof.

The reinforcement fibres may be pre-shaped fibres. The reinforcement fibres may be chopped or continuous, random or oriented, woven or non-woven, knitted or non-knitted or braided according to the requirements of any of various different portions of the desired structure of the composite material (also called fibre reinforced part) or the desired construction material (also called foundry shape).

The pre-shaped form of the reinforcement fibres may be selected in view of the desired form of e.g. the composite material, the fibre may have the form of a sheet, mat, bead, strand, thread, band, web, roving, band of rovings, bundle, or the like.

The amount of reinforcement fibres may vary depending on the desired need of e.g. the composite material. Reinforcement fibre content in the composition typically is in the range of up to 50 or even up to 80 wt % of the total weight of the composition, in another embodiment, the content of the reinforcement fibre may vary from 0.1 to 50 or even to 80 wt %, or from 1 to 50 or even to 80 wt %, or from 5 to 50 or even to 80 wt %, or from 10 to 50 or even to 80 wt %, or from 20 wt % to 50 or even to 80 wt % of the total weight of the composition.

In one embodiment, the filler is selected from the group consisting of organic fillers, preferably thermoplastics and elastomers; inorganic fillers, preferably glass microspheres, graphite or silica; and mineral powder fillers, preferably CaCO₃, coated CaCO₃, kaolin clay, SiO₂ (e.g. sand), talc, graphite, corundum (α-Al₂O₃), wollastonite, SiC, glass microspheres, mica, calcium silicate (Ca₂O₄Si), MgO, anhydrous calcium sulfate (CaSO₄ or anhydrite), ceramic hollow microspheres, fused mullite (Al₂O₃—SiO₂), boron nitride (BN), vermiculite, or basalt; and mixtures thereof. Preferably, the filler is sand.

In a preferred embodiment, the filler is selected from the group consisting of CaCO₃, coated CaCO₃, kaolin clay, SiO₂, wollastonite, boron nitride (BN), talc, and mixtures thereof.

In another preferred embodiment, the filler is selected from the group consisting of coated CaCO₃, talc, boron nitride (BN), wollastonite, SiC and mixtures thereof.

The fillers may be in particle, powder, sphere, chip and/or strand form and have an average particle size from nano scale to millimeters, preferably the fillers have an average particle size from 0.1 to 1000 μm, more preferably the fillers have an average particle size of from 0.5 to 500 μm.

The amount of fillers may vary and is preferably from 5 to 70 wt %, preferably from 15 to 50 wt %, more preferably from 15 to 45 wt %, based on the total weight of the composition (excluding any potential solvent).

In one embodiment, the composition comprises an additive (A) suitable for adhesive, coating, or 3D-printing applications. In this connection, suitable additives (A) are compounds comprising at least one α,β-unsaturated carbonyl group, epoxies, vinyl compounds, oxetanes, amines, isocyanates, and combinations thereof.

α,β-unsaturated carbonyl groups include vinyl ketones, acrylates, and acrylamides. In one embodiment, the composition comprises a compound comprising at least two α,β-unsaturated carbonyl groups. In one embodiment, the compound comprising at least two α,β-unsaturated carbonyl groups comprises at least two groups selected from the group consisting of vinyl ketones, acrylates, acrylamides, and combinations thereof.

Compounds comprising at least one α,β-unsaturated carbonyl group are suitably acrylates.

Suitable compounds comprising at least one α,β-unsaturated carbonyl group include alkyl acrylate such as methyl acrylate and compounds comprising at least two α,β-unsaturated carbonyl groups include ethanediol diacrylate, 1,3-propanediol diacrylate, 1,4-butanediol acrylate, poly(butanediol)diacrylate, polybutadiene diacrylate, 3-methyl-1,5-pentanediol diacrylate, 1,6-hexanediol diacrylate, ethylene or propylene glycol diacrylate, diethylene or dipropylene glycol diacrylate, triethylene or tripropylene glycol diacrylate, tertraethylene or tetrapropylene glycol diacrylate, polyethylene or polypropylene glycol diacrylate, resorcinol diglycidyl ether diacrylate, neopentyl glycol diacrylate, cyclohexane dimethanol diacrylate, ethoxylated or propoxylated neopentyl glycol diacrylate, ethoxylated or propoxylated cyclohexanedimethanol diacrylate, acrylated epoxy diacrylate, aryl and aliphatic urethane diacrylate polyester diacrylate, trimethylolpropane triacrylate, glycerol triacrylate, ethoxylated or propoxylated trimethylolpropane triacrylate, tris (2-hydroxyethyl) isocyanurate triacrylate, ethoxylated or propoxylated glycerol triacrylate, pentaerythritol triacrylate, aryl and aliphatic urethane triacrylates, melamine triacrylates, epoxy novolac triacrylates, pentaerythritol tetraccrylate, ethoxylated or propoxylated pentaerythritol tetraacrylate, dipentaerythritol tetraacrylate, ethoxylated or propoxylated dipentaerythritol tetraacrylate, aryl and aliphatic urethane tetraacrylates, melamine tetraacrylates, epoxy novolac tetraacrylates, and combinations thereof.

Suitable epoxies are selected from the group consisting of bisphenol A diglycidyl ether resins, bisphenol F diglycidyl ether resins, N,N,O-triglycidyl-3-aminophenol, N,N,O-triglycidyl-4 aminophenol, N,N,N′,N′ tetraglycidyl-4,4′-methylenebisbenzenamine, 4,4′,4″ meth-ylidene-trisphenol triglycidyl ether resins, naphthalenediol diglycidyl ethers, and mixtures thereof.

Suitable vinyl compounds include vinyl alcohol, styrene and derivatives thereof.

Amines may be an aromatic and/or an aliphatic amine. Suitably, the amine is a primary amine. Suitable primary amines include di- and polyamines, i.e. amines which include at least two primary amine groups.

In one embodiment, the primary amine is selected from aliphatic amines, cycloaliphatic amines, cycloaromatic amines, and combinations thereof. In one embodiment, the primary amine is selected from cycloaliphatic amines, cycloaromatic amines, and combinations thereof. The aliphatic amines, cycloaliphatic amines, cycloaromatic amines suitably include at least two primary amine groups, such as two primary amine groups (“diamines”), three primary amine groups (“triamines”), or four primary amine groups (“tetraamines”).

The term “isocyanate” as used herein encompasses any compound comprising at least one isocyanate functionality. Suitable isocyanates may further comprising an alkyl group, an alkylene group, a (optionally bivalent) cycloalkyl group, or a (optionally bivalent) aromatic group. Isocyanates may comprise more than one isocyanate functionality, e.g. diisocyanates such as diphenylmethane 4,4′-diisocyanate, isophorondiisocyanat, hexamethylendiisocyanat, and prepolymers thereof or triisocyanates.

In one embodiment, R¹⁰ is H, OH, C1-C8-alkyl, C1-C8-haloalkyl, C1-C8-alkoxy, C2-C8-alkenyl, C2-C8-alkynyl, C3-C8-cycloalkyl, C3-C8-cycloalkenyl, C6-C10-aryl, C6-C10-aryloxy, or OR^{A}; preferably OH, C1-C6-alkyl, C1-C6-haloalkyl, C1-C6-alkoxy, C2-C6-alkenyl, C2-C6-alkynyl, C3-C6-cycloalkyl, C3-C6-cycloalkenyl, C6-C10-aryl, C6-C10-aryloxy, or OR^{A}; more preferably OH, C1-C4-alkyl, C1-C4-haloalkyl, C1-C4-alkoxy, C2-C4-alkenyl, C2-C4-alkynyl, C3-C6-cycloalkyl, C3-C6-cycloalkenyl, phenyl, phenoxy, or OR^{A}.

In one embodiment, R¹⁰ is H, OH, C1-C8-alkyl, C1-C8-haloalkyl, C1-C8-alkoxy, C2-C8-alkenyl, C2-C8-alkynyl, or OR^{A}; preferably H, OH, C1-C6-alkyl, C1-C6-haloalkyl, C1-C6-alkoxy, C2-C6-alkenyl, C2-C6-alkynyl, or OR^{A}; more preferably H, OH, C1-C4-alkyl, C1-C4-haloalkyl, C1-C4-alkoxy, C2-C4-alkenyl, C2-C4-alkynyl, or OR^{A}.

In one embodiment, R¹⁰ is H, OH, C1-C8-alkyl, C1-C8-alkoxy, C2-C8-alkenyl, C2-C8-alkynyl, or OR^{A}; preferably H, OH, C1-C6-alkyl, C1-C6-alkoxy, C2-C6-alkenyl, C2-C6-alkynyl, or OR^{A}; more preferably H, OH, C1-C4-alkyl, C1-C4-alkoxy, C2-C4-alkenyl, C2-C4-alkynyl, or OR^{A}.

In a preferred embodiment, R¹⁰ is H, OH, C1-C6-alkyl, or OR^{A}; preferably is H, OH, C1-C5-alkyl, or OR^{A}; more preferably is H, OH, C1-C4-alkyl, or OR^{A}; even more preferably is H, OH, C1-C3-alkyl, or OR^{A}; and in particular H, OH, C1-C2-alkyl or OR^{A}.

In one embodiment, R¹⁰ is H.

In one embodiment, R¹⁰ is H or C1-C8-alkyl; preferably H or C1-C6-alkyl; more preferably H or C1-C4-alkyl; even more preferably H or C1-C3-alkyl; and in particular H or methyl.

In one embodiment, the compound of formula (10) or (10a) is the compound of formula (1) or (1a) according to the first aspect (including all embodiments).

In a preferred embodiment, the composition comprises at least one additive (A) selected from the group consisting of catalyst (C2), reinforcement fibres, and a filler.

In one embodiment, the composition comprises at least a catalyst (C2).

The composition may further comprise ingredients selected from the group pigments, compounds comprising at least one maleimide functionality, diene resins, and combinations thereof.

Suitable pigments include inorganic pigments such as titanium dioxide and zinc dioxide, and organic pigments such as anthraquinone pigments, anthrathrone pigments, anthrapyrimidine pigments, azo pigments, azomethine pigments, quinacridone pigments, quinophthalone pigments, diketopyrrolopyrrol pigments, inanthrone pigments, isoindoline pigments, metal complex pigments, perinone pigments, perylene pigments, phthalocyanine pigments, pyranthrone pigments, pyrazolo-quinazolone pigments, and thioindigo pigments.

Compounds comprising at least one maleimide functionality may comprise two maleimide functionalities, i.e. that the compound is a bismaleimide. Suitable bismaleimides include 4,4′-Diphenylmethane-bismaleimide, Bisallylnadic imide P, 4,4′-Diallylether bisphenol A, 2,2′-Diallyl bisphenol A (DABA), and 3,3′-dimethyl-5,5′-diethyl-4,4′-diphenylmethane bismaleimide.

Suitable diene resins include butadiene homopolymers, butadiene styrene copolymers, and maleinized polybutadienes.

As aforementioned, the present invention further relates in a fourth aspect to a process of manufacturing a 3D-printed object, a coating, a composite or construction material comprising the steps of
i) contacting a composition comprising a compound of formula (10) as defined in in the third aspect with an additive (A) selected from the group consisting of catalyst (C2), reinforcement fibres, a filler, compounds comprising at least one α,β-unsaturated carbonyl group, epoxies, vinyl compounds, oxetanes, amines, isocyanates, and combinations thereof to provide a mixture (M); and
ii) curing said mixture (M).

Particular embodiments (e.g. regarding x, y, Z, m, n, R¹, R¹⁰, R^{A}, R^{B}, p, and t, catalyst (C2), reinforcement fibres, filler, compounds comprising at least one α,β-unsaturated carbonyl group, epoxies, vinyl compounds, oxetanes, amines, isocyanates, and their amounts) are already above-outlined in connection with the first to third aspect and shall hold for the fourth aspect, as well. In the following, particular embodiments of the fourth aspect are described in further detail. It is to be understood that each embodiment is relevant on its own as well as in combination with other embodiments.

Depending to the desired properties of the composite or construction material the curing may be divided into at least two stages. In this connection, a first curing stage may aim at the acetoacetylated functionality and the second curing stage may aim at the cyclic ether functionality or vice versa.

In one embodiment, the compound of formula (10) is a compound of formula (10a) as defined in the third aspect.

In one embodiment, the invention relates to a process of manufacturing a composite or construction material comprising the steps of
i) contacting a fibre or a filler with a composition comprising a compound of formula (10) or (10a) as defined in in the third aspect to provide a fibre composition or a foundry mix, respectively; and
ii) curing said fibre composition or foundry mix, respectively.

In one embodiment, the process relates to the manufacturing of a composite material comprising the steps of
i) contacting a fibre with a composition comprising a compound of formula (10) or (10a) as defined in in the third aspect to provide a fibre composition, and
ii) curing said fibre composition.

In one embodiment, the fibres are reinforcement fibres.

In one embodiment, the contacting is an impregnation of the fibres with said composition.

Preparation of the composite materials according to the invention can be achieved as follows: providing a compound of formula (10) or (10a) and optionally a catalyst (C2), intimately mixing the components together as necessary, casting the mixture into the desired form comprising reinforcement fibres, and then initiating polymerization of the mixture (e.g. by, inter alia, increasing temperature, irradiation (e.g. UV light), use of a catalyst (C2), or other methods commonly known in the art).

In one embodiment, the process comprises:
(i) providing the compound of formula (10) or (10a) and optionally adding a catalyst (C2) and/or a solvent to said compound of formula (10) or (10a) to obtain a mixture (by intimately mixing), preferably a homogenous mixture;
(ii) providing a fibre structure;
(iii) placing said fibre structure in a mold or on a substrate;
(iv) impregnating said fibre structure with said mixture (from step (i)), optionally by applying elevated pressure and/or evacuating the air and solvent from the mold and fibre structure, preferably at a temperature of 50 to 150° C.; and
curing said liquid mixture in cured laminates by applying a temperature of preferably 50 to 180° C. with heating steps for a time sufficient to achieve a degree of conversion that allows de-moulding of the parts.

In another embodiment, the impregnation in step (iv) is achieved using a method selected from the group consisting of pre-preg (hot melt and solvated), resin transfer molding, vacuum assisted resin transfer molding, Vacuum resin infusion, Seemann Composites Resin Infusion Molding Process, injection molding, compression molding, spray molding, pultrusion, hand laminating, filament winding, Quickstep process or Roctool process.

In another embodiment, the impregnation in step (iv) is achieved using a composite molding process method selected from the group consisting of pre-preg (hot melt and solvated), resin transfer molding, liquid resin infusion, Seemann Composites Resin Infusion Molding Process, vacumn assisted resin infusion, injection molding, BMC/SMC bulk and sheet molding compounds and EADS vacuum assisted process (VAP®).

Fibre content in the fibre composition or in the fibre reinforced parts typically is in the range of up to 50 or even up to 80 wt % of the total weight of the fibre resin composition or of the fibre reinforced part respectively, in another embodiment, the content of the fibre may vary from 0.1 to 50 or even to 80 wt %, or from 1 to 50 or even to 80 wt %, or from 5 to 50 or even to 80 wt %, or from 10 to 50 or even to 80 wt %, or from 20 wt % to 50 or even to 80 wt % of the total weight of the fibre resin composition or of the fibre reinforced part respectively.

In another embodiment, the process relates to the manufacturing of a construction material comprising the steps of
i) mixing a filler with a composition comprising a compound of formula (10) or (10a) as defined in the third aspect to give a foundry mix, and
ii) curing said foundry mix.

In one embodiment, the filler is sand. The sand for use according to the process of the invention may be any sand that is suitable for use as a filler. Examples include silica sand, olivine sand, chromite sand, zircon sand, chamotte sand. The sand may comprise minor amounts of staurolite, graphite or coal, clay, talc, iron oxides, titanium oxides and/or anthracite. The clay may be kaolin and/or bentonite. The sand may further comprise cushioning material such as wood flour, saw dust, powdered husks, peat, and straw and/or cereal binders such as dextrin, starch, sulphite lye, and molasses.

The amount of the composition comprising a compound of formula (10) or (10a) as defined in the third aspect relative to the amount of the filler (i.e., sand) in the foundry mix may vary depending on the desired application, but is generally from 0.5% (w/w) to 15% (w/w), preferably from 1% (w/w) to 12.5% (w/w), more preferably from 2% (w/w) to 10% (w/w), especially from 1% (w/w) to 10% (w/w), more especially from 1% (w/w) to 2% (w/w), most especially from 0.5% (w/w) to 3% (w/w).

In one embodiment, the process relates to the manufacturing of a 3D-printed object comprising the curing steps repeatedly. Any known in the art 3D-printing method is suitable.

In one embodiment, the process relates to the manufacturing of a coating, wherein the process further comprises a coating step. The coating step is preferably performed between the step of i) contacting and the step of ii) curing.

As aforementioned, the present invention further relates in a fifth aspect to a 3D-printed object, a coating, a composite or construction material obtainable by a process according to the fourth aspect.

Particular embodiments (e.g. regarding x, y, Z, m, n, R¹, R¹⁰, R^{A}, R^{B}, p, and t, catalyst (C2), reinforcement fibres, filler, compounds comprising at least one α,β-unsaturated carbonyl group, epoxies, vinyl compounds, oxetanes, amines, isocyanates, and their amounts) are already above-outlined in connection with the first to fourth aspect and shall hold for the composite and construction material, as well. In the following, particular embodiments of the fifth aspect are described in further detail. It is to be understood that each embodiment is relevant on its own as well as in combination with other embodiments.

Depending on the field of application, the glass transition temperature (Tg) of the product can be adjusted. Preferably, the composite or construction material has a glass transition temperature (Tg) of about 40 to about 200° C., preferably of about 50 to about 180° C. Preferably, the coating has a glass transition temperature (Tg) of about −90 to about 80° C., preferably of about −70 to about 70° C. such as of about −50 to about 60° C.

In one embodiment, the 3D-printed object, the coating, the composite or construction material is obtained by a process according to the fourth aspect.

As aforementioned, the present invention further relates in a sixth aspect to the use of a compound according to the first aspect or to a composition according to the third aspect in 3D-pritning or for producing a coating, an adhesive, a composite or a construction material.

Particular embodiments (e.g. regarding x, y, Z m, n, R¹, R¹⁰, R^{A}, R^{B}, p, and t, catalyst (C2), reinforcement fibres, filler, compounds comprising at least one α,β-unsaturated carbonyl group, epoxies, vinyl compounds, oxetanes, amines, isocyanates, and their amounts) are already above-outlined in connection with the first to fifth aspect and shall hold for the use, as well.

Depending on the field of application, the glass transition temperature (Tg) of the product can be adjusted. Preferably, the composite or construction material has a glass transition temperature (Tg) of about 40 to about 200° C., preferably of about 50 to about 180° C. Preferably, the coating has a glass transition temperature (Tg) of about −90 to about 80° C., preferably of about −70 to about 70° C. such as of about −50 to about 60° C.

The skilled person in the art will be aware of how to use the respective ingredients for 3D-printing or for manufacturing a coating, an adhesive, a composite or a construction material.

In one embodiment, the compound according to the first aspect or the composition according to the third aspect are used for producing a coating, wherein the coating comprises polyurea, polyurethane, and combinations thereof or wherein the coating comprises condensation products of epoxies and amines.

In one embodiment, the coating is a paint. Such paint suitably comprise at least one pigment.

In one embodiment, the compound of formula (1) or (1a) is used for producing a coating comprising polyurea, polyurethane, and combinations thereof.

In one embodiment, the compound of formula (1) or (1a) is used for producing a coating comprising condensation products of epoxies and amines.

In one embodiment, the composition comprising a compound of formula (10) or (10a) is used for producing a coating comprising polyurea, polyurethane, and combinations thereof.

In one embodiment, the composition comprising a compound of formula (10) or (10a) is used for producing a coating comprising condensation products of epoxies and amines.

As aforementioned, the present invention further relates in a seventh aspect to a method for fastening anchoring elements in boreholes, wherein a composition according to the third aspect is placed in the boreholes and the anchoring element is inserted therein.

Particular embodiments (e.g. regarding x, y, Z, m, n, R¹, R¹⁰, R^{A}, R^{B}, p, and t, catalyst (C2), reinforcement fibres, filler, compounds comprising at least one α,β-unsaturated carbonyl group, epoxies, vinyl compounds, oxetanes, amines, isocyanates, and their amounts) are already above-outlined in connection with the first to fourth aspect and shall hold for the seventh aspect, as well. In the following, particular embodiments of the seventh aspect are described in further detail. It is to be understood that each embodiment is relevant on its own as well as in combination with other embodiments.

Suitable anchoring elements include tie bars and dowels.

In one embodiment, the composition according to the third aspect comprises a catalyst (C2).

As aforementioned, the present invention further relates in an eighth aspect to a 3D printing resin composition comprising a compound of formula (10)

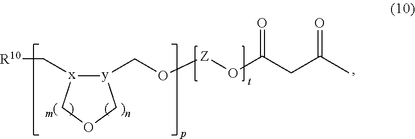

wherein
R¹⁰ is H, OH, C1-C10-alkyl, C1-C10-haloalkyl, C1-C10-alkoxy, C2-C10-alkenyl, C2-C10-alkynyl, C3-C10-cycloalkyl, C3-C10-cycloalkenyl, C3-C10-aryl, C3-C10-aryloxy, or OR^{A};
Z is within each unit independently C1-C10-alkylene, C2-C10-alkenylene, C2-C10-alkynylene, C3-C10 bivalent aryl, C(O)—C3-C10-aryl-C(O), C1-C10-alkylene-C3-C10-aryl-C1-C10-alkylene, or C3-C10-aryl-C1-C10-alkylene-C3-C10-aryl, wherein each carbon atom of the aforementioned substituents may independently be unsubstituted or substituted with one or more same of different of R^{B};
within each unit independently x and y are either both CH or x is C and y is not present so that the respective unit corresponds to a spiro cyclic moiety of formula (SC)

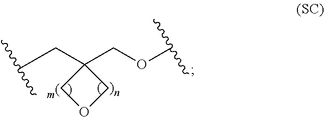

m is independently 0, 1, or 2;
n is independently 0, 1, or 2;
wherein within one unit when y is not present at least one of m or n is 1;
p is an integer of 1 to 6;
R^{A} is a moiety of formula (A-1) or formula (A-2)

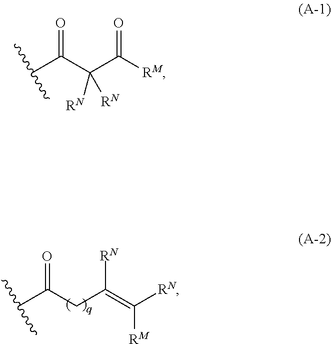

wherein
R^{N} is independently H, halogen, or C1-C3-alkyl;
R^{M} is C1-C6-alkyl or C1-C6-haloalkyl;
R^{B} is halogen, C1-C6-alkyl, or C1-C6-alkoxy;
t is 0 or an integer of 1 to 10000; and
q is 0 or an integer of 1 to 6.

Particular embodiments (e.g. regarding x, y, Z, m, n, R¹, R¹⁰, R^{A}, R^{B}, p, and t, catalyst (C2), reinforcement fibres, filler, compounds comprising at least one α,β-unsaturated carbonyl group, epoxies, vinyl compounds, oxetanes, amines, isocyanates, and their amounts) are already above-outlined in connection with the above aspects and shall hold for the eighth aspect, as well. In the following, particular embodiments of the eighth aspect are described in further detail. It is to be understood that each embodiment is relevant on its own as well as in combination with other embodiments.

In one embodiment, the compound of formula (10) is a compound of formula (10a) as defined in the third aspect.

In one embodiment, the 3D printing resin composition further comprises an additive (A). Potential additives (A) are already mentioned in connection with the third aspect.

The 3D printing resin composition is particularly suitable for digital light printing (DLP), continuous liquid interface printing 5 (CLIP), and/or Stereolithography (SL).

It will be obvious for a person skilled in the art that these embodiments only depict examples of a plurality of possibilities. Hence, the embodiments shown here should not be understood to form a limitation of these features and configurations. Any possible combination and configuration of the described features can be chosen according to the scope of the invention.

The present invention will be further illustrated by the following examples.

### EXAMPLES

### Chemicals

| | | Purchased |
|---|---|---|
| Abbreviation | Chemical details | from |
| EHO | 3-ethyl-3-hydroxymethyl-oxetane | Merck |
| DABCO | 1,4-diazabicyclo[2.2.2]octane | Merck |
| Suprasec | prepolymerised diphenyl methane | Huntsman |
| 2008 | diisocyanate (CAS 101-68-8) | |
| DETDA 80 | 3,5-Diethyl-2,4(2,6)-toluoldiamin | Arxada |
| | (CAS 68479-98-1) | |
| Epikote 828 | epoxy resin | Hexion |
| TMPTA | 2,2-Bis[(acryloyloxy)methyl]butyl | Merck |
| | acrylate (CAS 15625-89-5) | |
| AATMP | Trimethylolpropan triacetoacetat | Arxada |
| | (CAS 22208-25-9) | |
| DBU | 1,8-Diazabicyclo[5.4.0]-7-undecene | Merck |
| | (CAS 6674-22-2) | |

### Material

Mettler Toledo—Thermal Mechanical Analysis (TMA SDTA840); used temperature cycle 25° C. to 100° C. with 10K/min.

### Example 1

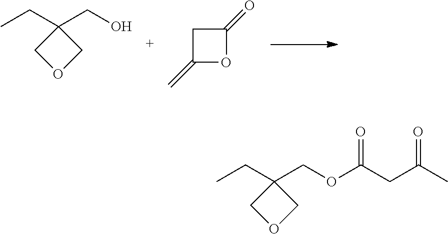

Under inert gas atmosphere, 40.0 g (0.33 mol, 1 eq.) 3-ethyl-3-hydroxymethyl-oxetane (EHO) and 19.1 mg (0.17 mmol, 0.0005 eq.) 1,4-diazabicyclo[2.2.2]octane (DABCO) were charged into a 250 mL reactor and the mixture was heated to 30° C. A total of 28.9 g (0.34 mol, 1.04 eq.) diketene was added over 2.5 h under temperature control. After cooling to room temperature the final product was obtained in a yield of 100% and purity >99% (wt % by 1H NMR).

### Example 2

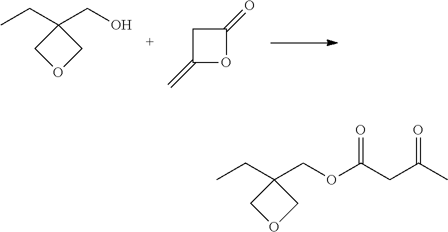

Under inert gas atmosphere, 20.0 g (0.17 mol, 1 eq.) 3-ethyl-3-hydroxymethyl-oxetane (EHO), 21.0 g ethyl acetate and 9.56 mg (0.08 mmol, 0.0005 eq.) 1,4-diazabicyclo[2.2.2]octane (DABCO) were charged into a 250 mL reactor and the mixture was heated to 30° C. A total of 14.5 g (0.17 mol, 1.04 eq.) diketene was added over 1 h under temperature control. After concentration of the reaction mixture by vacuum distillation at 30° C. the final product was obtained in a yield of 100% and purity >98% (wt % by 1H NMR).

### Application Example—Michael Addition Chemistry

0.36 g (0.0018 mol) of product from Example 1 was mixed with 3.3 g (0.0085 mol) AATMP and 40 μl DBU at rt (room temperature). To this solution, 6.3 g (0.021 mol) TMPTA is added and the material is homogenized. Then liquid material is transferred into an aluminum mold and allowed to cure at rt for 24 h. A hard disc with a Tg (TMA) of 45-55° C. was obtained. 0.7 g (0.0034 mol) of product from Example 1 was mixed with 3 g (0.0085 mol) AATMP and 40 μl DBU at room temperature. To this solution, 6.3 g (0.021 mol) TMPTA is added and the material is homogenized. Then liquid material is transferred into an aluminum mold and allowed to cure at rt for 24 h. A hard disc with a Tg (TMA) of 45-60° C. was obtained.

### Application Example—Reaction with Isocyante

0.7 g (0.0034 mol) of product from Example 1 and 0.7 g DETDA 80 was mixed with 3 g Suprasec 2008 Isocyanate at room temperature. With the help of a drawdown bar a film layer of about 200 μm thickness was created. The film was then allowed to dry at rt to obtain a solid coating film.

### Application Example—Reaction with Epoxy

0.7 g (0.0034 mol) of product from Example 1 and 1.1 g DETDA 80 was mixed with 3 g Epikote 828 Epoxy. With the help of a drawdown bar a film layer of about 200 μm thickness was created. The film was then allowed to dry to obtain a solid coating film.

The invention further relates to the following items:

1. A compound of formula (1)

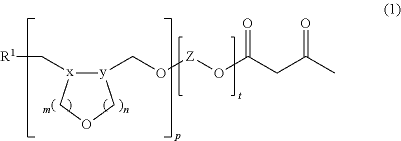

wherein
R¹ is OH, C1-C10-alkyl, C1-C10-haloalkyl, C1-C10-alkoxy, C2-C10-alkenyl, C2-C10-alkynyl, C3-C10-cycloalkyl, C3-C10-cycloalkenyl, C3-C10-aryl, C3-C10-aryloxy, or OR^{A};
Z is within each unit independently C1-C10-alkylene, C2-C10-alkenylene, C2-C10-alkynylene, C3-C10 bivalent aryl, C(O)—C3-C10-aryl-C(O), C1-C10-alkylene-C3-C10-aryl-C1-C10-alkylene, or C3-C10-aryl-C1-C10-alkylene-C3-C10-aryl, wherein each carbon atom of the aforementioned substituents may independently be unsubstituted or substituted with one or more same of different of R^{B};
within each unit independently x and y are either both CH or x is C and y is not present so that the respective unit corresponds to a spiro cyclic moiety of formula (SC)

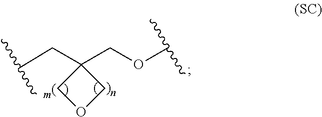

m is independently 0, 1, or 2;
n is independently 0, 1, or 2;
wherein within one unit when y is not present at least one of m or n is 1;
p is an integer of 1 to 6;
R^{A} is a moiety of formula (A-1) or formula (A-2)

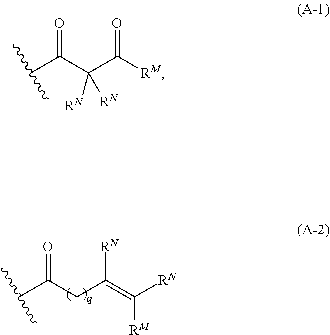

wherein
R^{N} is independently H, halogen, or C1-C3-alkyl;
R^{M} is C1-C6-alkyl or C1-C6-haloalkyl;
R^{B} is halogen, C1-C6-alkyl, or C1-C6-alkoxy;
t is 0 or an integer of 1 to 10000; and
q is 0 or an integer of 1 to 6.

2. The compound according to item 1, wherein
R¹ is C1-C8-alkyl, C1-C8-alkoxy, C2-C8-alkenyl, or OR^{A}; preferably C1-C6-alkyl, C1-C6-alkoxy, C2-C6-alkenyl, or OR^{A}; more preferably C1-C4-alkyl, C1-C4-alkoxy, C2-C4-alkenyl, or OR^{A}; and/or m and n are independently 0 or 1; and/or;
p is an integer of 1 to 3.

3. The compound according to item 1 or 2, wherein
each x is C and each y is not present so that the compound of formula (1) is a compound of formula (1-2a)

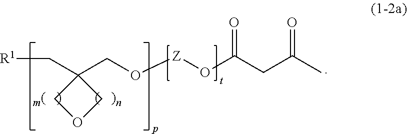

4. The compound according to item 1, wherein
R¹ is C1-C6-alkyl, preferably C1-C3-alkyl, more preferably methyl;
x is C and y is not present;
t is 0 or an integer of 1 to 10000, preferably 0 or 1 more preferably 0; and
p is 1,
preferably wherein m and n are 1.

5. The compound according to item 1, wherein
R¹ is C1-C3-alkyl or OR^{A};
p is 1 or 2;
R^{A} is a moiety of formula (A-1) or formula (A-2)

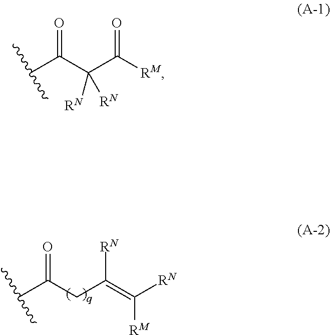

wherein
R^{N} is independently H or methyl, preferably H;
R^{M} is 1-C3-alkyl, preferably methyl; and
q is 0 or an integer of 1 to 3, preferably 0,
preferably wherein each x is C and each y is not present.

6. A process of manufacturing an acetoacetylated alcohol, the process comprising the step of reacting an alcohol of formula (20)

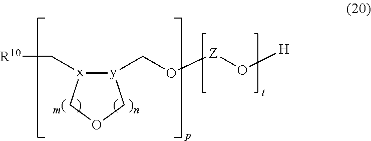

in the presence of a catalyst (C1) with diketene, wherein
R¹⁰ is H, OH, C1-C10-alkyl, C1-C10-haloalkyl, C1-C10-alkoxy, C2-C10-alkenyl, C2-C10-alkynyl, C3-C10-cycloalkyl, C3-C10-cycloalkenyl, C3-C10-aryl, C3-C10-aryloxy, or OR^{A};
Z is within each unit independently C1-C10-alkylene, C2-C10-alkenylene, C2-C10-alkynylene, C3-C10 bivalent aryl, C(O)—C3-C10-aryl-C(O), C1-C10-alkylene-C3-C10-aryl-C1-C10-alkylene, or C3-C10-aryl-C1-C10-alkylene-C3-C10-aryl, wherein each carbon atom of the aforementioned substituents may independently be unsubstituted or substituted with one or more same of different of R^{B};
within each unit independently x and y are either both CH or x is C and y is not present so that the respective unit corresponds to a spiro cyclic moiety of formula (SC)

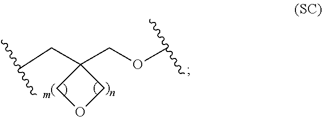

m is independently 0, 1, or 2;
n is independently 0, 1, or 2;
wherein within one unit when y is not present at least one of m or n is 1;
p is an integer of 1 to 6;
R^{A} is a moiety of formula (A-1) or formula (A-2)

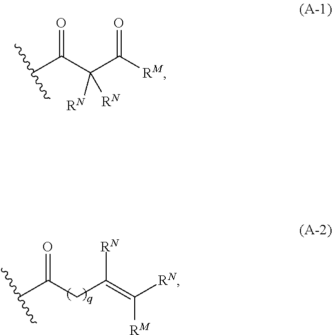

wherein
R^{N} is independently H, halogen, or C1-C3-alkyl;
R^{M} is C1-C6-alkyl or C1-C6-haloalkyl;
R^{B} is halogen, C1-C6-alkyl, or C1-C6-alkoxy;
t is 0 or an integer of 1 to 10000; and
q is 0 or an integer of 1 to 6.

7. The process according to item 6, wherein the acetoacetylated alcohol is a compound of formula (10)

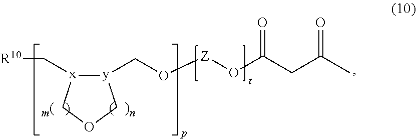

wherein
R¹⁰ is H, OH, C1-C10-alkyl, C1-C10-haloalkyl, C1-C10-alkoxy, C2-C10-alkenyl, C2-C10-alkynyl, C3-C10-cycloalkyl, C3-C10-cycloalkenyl, C3-C10-aryl, C3-C10-aryloxy, or OR^{A};
Z is within each unit independently C1-C10-alkylene, C2-C10-alkenylene, C2-C10-alkynylene, C3-C10 bivalent aryl, C(O)—C3-C10-aryl-C(O), C1-C10-alkylene-C3-C10-aryl-C1-C10-alkylene, or C3-C10-aryl-C1-C10-alkylene-C3-C10-aryl, wherein each carbon atom of the aforementioned substituents may independently be unsubstituted or substituted with one or more same of different of R^{B};
within each unit independently x and y are either both CH or x is C and y is not present so that the respective unit corresponds to a spiro cyclic moiety of formula (SC)

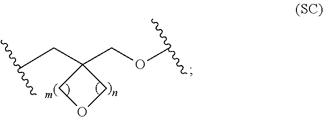

m is independently 0, 1, or 2;
n is independently 0, 1, or 2;
wherein within one unit when y is not present at least one of m or n is 1;
p is an integer of 1 to 6;
R^{A} is a moiety of formula (A-1) or formula (A-2)

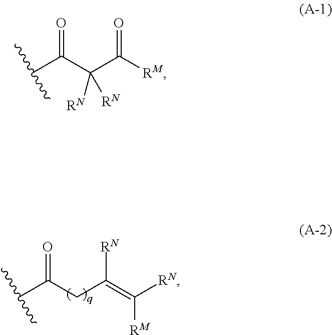

wherein
R^{N} is independently H, halogen, or C1-C3-alkyl;
R^{M} is C1-C6-alkyl or C1-C6-haloalkyl;
R^{B} is halogen, C1-C6-alkyl, or C1-C6-alkoxy;
t is 0 or an integer of 1 to 10000; and
q is 0 or an integer of 1 to 6,
preferably wherein the acetoacetylated alcohol is a compound of formula (1) according to any one of items 1 to 5.

8. The process according to item 6 or 7, wherein the catalyst (C1) is a tertiary amine, preferably selected from the group consisting of trialkyl amine, 1,4-diazabicyclo[2.2.2]octane (DABCO), N,N-dimethylpyridin-4-amin, 1,1,3,3-tetramethylguanidine (TMG), N,N-dimethylisopropylamine, 1,8-diazabicyclo(5.4.0)undec-7-ene (DBU), 1,5-diazabicyclo(4.3.0)non-5-ene (DBN), resin bond teriary amines, and combinations thereof, and/or carboxylic acid salts, preferably selected form the group consisting of sodium acetate, potassium acetate, and combinations thereof.

9. The process according to any one of items 6 to 8, wherein the diketene and the alcohol of formula (20) are present in the reaction in a molar ratio of about 1:2 to about 10:1, preferably of about 1:1.5 to about 6:1, more preferably of about 1:1.3 to about 3:1, and in particular of about 1:1.1 to about 2.1:1.

10. The process according to any one of items 6 to 9, wherein the reaction is performed in the absence of a solvent.

11. A composition comprising a compound of formula (10)

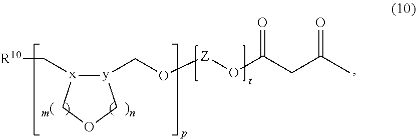

wherein
R¹⁰ is H, OH, C1-C10-alkyl, C1-C10-haloalkyl, C1-C10-alkoxy, C2-C10-alkenyl, C2-C10-alkynyl, C3-C10-cycloalkyl, C3-C10-cycloalkenyl, C3-C10-aryl, C3-C10-aryloxy, or OR^{A};
Z is within each unit independently C1-C10-alkylene, C2-C10-alkenylene, C2-C10-alkynylene, C3-C10 bivalent aryl, C(O)—C3-C10-aryl-C(O), C1-C10-alkylene-C3-C10-aryl-C1-C10-alkylene, or C3-C10-aryl-C1-C10-alkylene-C3-C10-aryl, wherein each carbon atom of the aforementioned substituents may independently be unsubstituted or substituted with one or more same of different of R^{B};
within each unit independently x and y are either both CH or x is C and y is not present so that the respective unit corresponds to a spiro cyclic moiety of formula (SC)

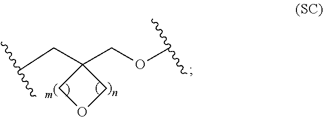

m is independently 0, 1, or 2;
n is independently 0, 1, or 2;
wherein within one unit when y is not present at least one of m or n is 1;
p is an integer of 1 to 6;
R^{A} is a moiety of formula (A-1) or formula (A-2)

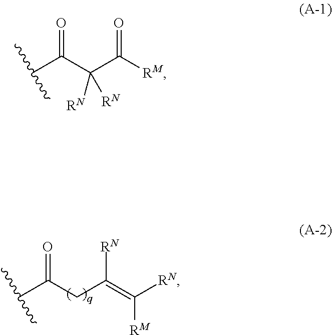

wherein
R^{N} is independently H, halogen, or C1-C3-alkyl;
R^{M} is C1-C6-alkyl or C1-C6-haloalkyl;
R^{B} is halogen, C1-C6-alkyl, or C1-C6-alkoxy;
t is 0 or an integer of 1 to 10000; and
q is 0 or an integer of 1 to 6,
and an additive (A) selected from the group consisting of catalyst (C2), reinforcement fibres, a filler, compounds comprising at least one α,β-unsaturated carbonyl group, epoxies, vinyl compounds, oxetanes, amines, isocyanates, and combinations thereof.

12. The composition according to item 11, wherein the catalyst (C2) is selected from the group consisting of aliphatic mono-, di- and polyamines; aromatic mono-, di- and polyamines; carbocyclic mono-, di and polyamines; heterocyclic mono-, di- and polyamines; compounds containing a five- or six-membered nitrogen-containing heterocyclic ring; hydroxyamines; phosphines; phenols; and mixtures thereof, and/or
the reinforcement fibres are selected from the group consisting of carbon fibres; glass fibres, preferably E glass fibres or S glass fibres; aramid fibres; basalt fibres; natural fibres, preferably flax, hemp, jute or sisal; fleeces; and woven fabrics; and mixtures thereof, and/or
wherein the filler is selected from the group consisting of organic fillers, preferably thermoplastics and elastomers; inorganic fillers, preferably glass microspheres, graphite or silica; and mineral powder fillers, preferably CaCO₃, coated CaCO₃, kaolin clay, SiO₂, talc, graphite, corundum (α-Al₂O₃), wollastonite, SiC, glass microspheres, mica, calcium silicate (Ca₂O₄Si), MgO, anhydrous calcium sulfate (CaSO₄ or anhydrite), ceramic hollow microspheres, fused mullite (Al₂O₃—SiO₂), boron nitride (BN), vermiculite, or basalt; and mixtures thereof.

13. A process of manufacturing a 3D-printed object, a coating, an adhesive, a composite or construction material comprising the steps of
i) contacting a composition comprising a compound of formula (10) as defined in in item 11 with an additive (A) selected from the group consisting of catalyst (C2), reinforcement fibres, a filler, compounds comprising at least one α,β-unsaturated carbonyl group, epoxies, vinyl compounds, oxetanes, amines, isocyanates, and combinations thereof to provide a mixture (M); and
ii) curing said mixture (M).

14. A 3D-printed object, a coating, an adhesive, a composite or construction material obtainable by a process according to item 13.

15. Use of a compound according to any one of items 1 to 5 or of a composition according to any one of items 11 to 13 in 3D-printing or for producing a coating, an adhesive, a composite or construction material.

16. The use according to item 15 for producing a coating, wherein the coating comprises polyurea, polyurethane, and combinations thereof or wherein the coating comprises condensation products of epoxies and amines.

17. A method for fastening anchoring elements in boreholes, wherein a composition according to any one of items 11 to 13 is placed in the boreholes and the anchoring element is inserted therein.

18. A 3D printing resin composition comprising a compound of formula (10)

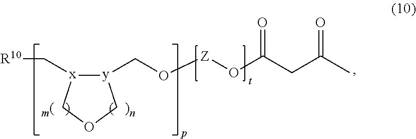

wherein
R¹⁰ is H, OH, C1-C10-alkyl, C1-C10-haloalkyl, C1-C10-alkoxy, C2-C10-alkenyl, C2-C10-alkynyl, C3-C10-cycloalkyl, C3-C10-cycloalkenyl, C3-C10-aryl, C3-C10-aryloxy, or OR^{A};
Z is within each unit independently C1-C10-alkylene, C2-C10-alkenylene, C2-C10-alkynylene, C3-C10 bivalent aryl, C(O)—C3-C10-aryl-C(O), C1-C10-alkylene-C3-C10-aryl-C1-C10-alkylene, or C3-C10-aryl-C1-C10-alkylene-C3-C10-aryl, wherein each carbon atom of the aforementioned substituents may independently be unsubstituted or substituted with one or more same of different of R^{B};
within each unit independently x and y are either both CH or x is C and y is not present so that the respective unit corresponds to a spiro cyclic moiety of formula (SC)

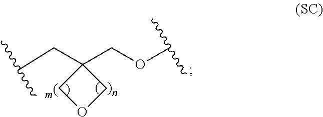

m is independently 0, 1, or 2;
n is independently 0, 1, or 2;
wherein within one unit when y is not present at least one of m or n is 1;
p is an integer of 1 to 6;
R^{A} is a moiety of formula (A-1) or formula (A-2)

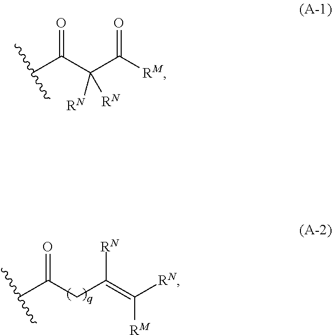

wherein
R^{N} is independently H, halogen, or C1-C3-alkyl;
R^{M} is C1-C6-alkyl or C1-C6-haloalkyl;
R^{B} is halogen, C1-C6-alkyl, or C1-C6-alkoxy;
t is 0 or an integer of 1 to 10000; and
q is 0 or an integer of 1 to 6.

1. A compound of formula 1(a) 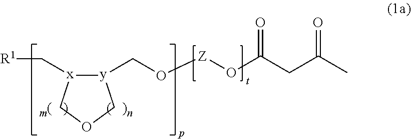
wherein R1 is OH, C1-C10-alkyl, C1-C10-haloalkyl, C1-C10-alkoxy, C2-C10-alkenyl, C2-C10-alkynyl, C3-C10-cycloalkyl, C3-C10-cycloalkenyl, C3-C10-aryl, C3-C10-aryloxy, or OR^{A};
Z is within each unit independently C1-C3-alkylene, wherein each carbon atom of the aforementioned substituents may independently be unsubstituted or substituted with one or more same of different of R^{B};
within each unit independently x and y are either both CH or x is Cand y is not present so that the respective unit corresponds to a spiro cyclic moiety of formula (SC) 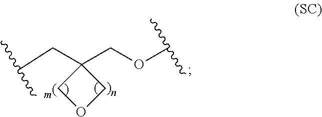
m is independently 0, 1, or 2;
n is independently 0, 1, or 2;
wherein within one unit when y is not present at least one of morn is 1;
p is an integer of 1 to 6;
R^{A} is a moiety of formula (A-1) or formula (A-2) 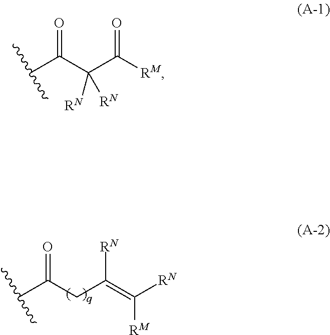
wherein R^{N} is independently H, halogen, or C1-C3-alkyl;
R^{M} is C1-C6-alkyl or C1-C6-haloalkyl;
R^{B} is halogen or methyl;
t is 0 or an integer of 1 to 2; and
q is O or an integer of 1 to 6.

2. The compound according to claim 1, wherein at least one of
R¹ is C1-C8-alkyl, C1-C8-alkoxy, C2-C8-alkenyl, or OR^{A};
m and n are independently O or 1; or;
p is an integer of 1 to 3.

3. The compound according to claim 1, wherein
each x is C and each y is not present so that the compound of formula (1 a) is a compound of formula (1-2a)

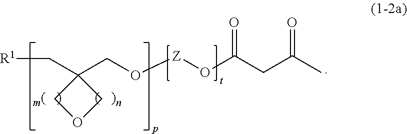

4. The compound according to claim 1, wherein
R1 is C1-C6-alkyl;
x is C and y is not present; and
t is O or an integer of 1 to 10000.

5. The compound according to claim 1, wherein
R1 is C1-C3-alkyl or OR^{A};
p is 1 or 2;
R^{A} is a moiety of formula (A-1) or formula (A-2) 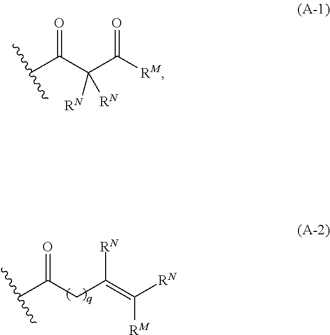
wherein
R^{N} is independently H or methyl; and
q is O or an integer of 1 to 3.

6. A process of manufacturing an acetoacetylated alcohol, the process comprising the step of reacting an alcohol of formula (20) 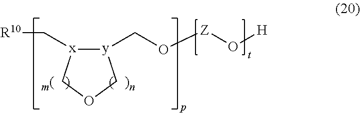
in the presence of a catalyst (C1) with diketene, wherein
R¹⁰ is H, OH, C1-C10-alkyl, C1-C10-haloalkyl, C1-C10-alkoxy, C2-C10-alkenyl, C2-C10-alkynyl, C3-C10-cycloalkyl, C3-C10-cycloalkenyl, C3-C10-aryl, C3-C10-aryloxy, or OR^{A};
Z is within each unit independently C1-C10-alkylene, C2-C10-alkenylene, C2-C10-alkynylene, C3-C10 bivalent aryl, C(O)—C3-C10-aryl-C(O), C1-C10-alkylene-C3-C10-aryl-C1-C10-alkylene, or C3-C10-aryl-C1-C10-alkylene-C3-C10-aryl, wherein each carbon atom of the aforementioned substituents may independently be unsubstituted or substituted with one or more same of different of R^{B};
within each unit independently x and y are either both CH or x is C and y is not present so that the respective unit corresponds to a spiro cyclic moiety of formula (SC) 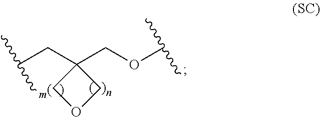
m is independently 0, 1, or 2;
n is independently 0, 1, or 2;
wherein within one unit when y is not present at least one of m or n is 1; p is an integer of 1 to 6;
R^{A} is a moiety of formula (A-1) or formula (A-2) 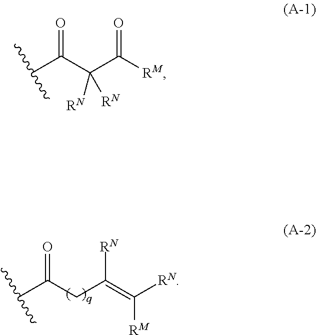
wherein
R^{N} is independently H, halogen, or C1-C3-alkyl;
R^{M} is C1-C6-alkyl or C1-C6-haloalkyl;
R^{B} is halogen, C1-C6-alkyl, or C1-C6-alkoxy;
t is 0 or an integer of 1 to 10000; and
q is 0 or an integer of 1 to 6.

7. The process according to claim 6, wherein the acetoacetylated alcohol is a compound of formula (10) 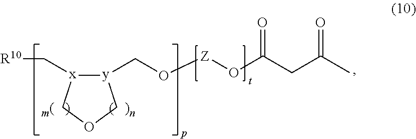
wherein
R¹⁰ is H, OH, C1-C10-alkyl, C1-C10-haloalkyl, C1-C10-alkoxy, C2-C10-alkenyl, C2-C10-alkynyl, C3-C10-cycloalkyl, C3-C10-cycloalkenyl, C3-C10-aryl, C3-C10-aryloxy, or OR^{A};
Z is within each unit independently C1-C10-alkylene, C2-C10-alkenylene, C2-C10-alkynylene, C3-C10 bivalent aryl, C(O)—C3-C10-aryl-C(O), C1-C10-alkylene-C3-C10-aryl-C1-C10-alkylene, or C3-C10-aryl-C1-C10-alkylene-C3-C10-aryl, wherein each carbon atom of the aforementioned substituents may independently be unsubstituted or substituted with one or more same of different of R^{B};
within each unit independently x and y are either both CH or x is C and y is not present so that the respective unit corresponds to a spiro cyclic moiety of formula (SC) 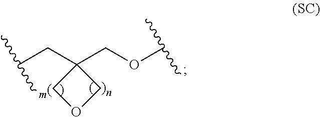
m is independently 0, 1, or 2;
n is independently 0, 1, or 2;
wherein within one unit when y is not present at least one of m or n is 1; p is an integer of 1 to 6;
R^{A} is a moiety of formula (A-1) or formula (A-2) 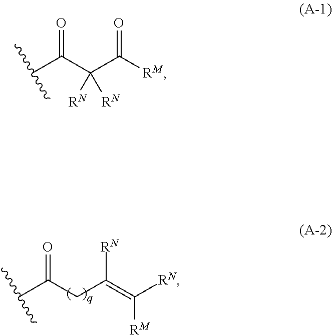
wherein
R^{N} is independently H, halogen, or C1-C3-alkyl;
R^{M} is C1-C6-alkyl or C1-C6-haloalkyl;
R⁸ is halogen, C1-C6-alkyl, or C1-C6-alkoxy;
t is O or an integer of 1 to 10000; and
q is O or an integer of 1 to 6.

8. The process according to claim 6, wherein the catalyst (C1) is one or more of a tertiary amine.

9. The process according to claim 6, wherein the diketene and the alcohol of formula (20) are present in the reaction in a molar ratio of about 1:2 to about 10:1.

10. The process according to claim 6, wherein the reaction is performed in the absence of a solvent.

11. A composition comprising a compound of formula (10) 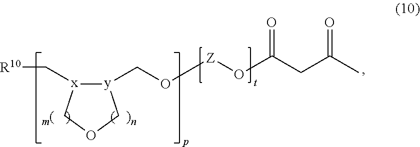
wherein
R¹⁰ is H, OH, C1-C10-alkyl, C1-C10-haloalkyl, C1-C10-alkoxy, C2-C10-alkenyl, C2-C10-alkynyl, C3-C10-cycloalkyl, C3-C10-cycloalkenyl, C3-C10-aryl, C3-C10-aryloxy, or OR^{A};
Z is within each unit independently C1-C10-alkylene, C2-C10-alkenylene, C2-C10-alkynylene, C3-C10 bivalent aryl, C(O)—C3-C10-aryl-C(O), C1-C10-alkylene-C3-C10-aryl-C1-C10-alkylene, or C3-C10-aryl-C1-C10-alkylene-C3-C10-aryl, wherein each carbon atom of the aforementioned substituents may independently be unsubstituted or substituted with one or more same of different of R^{B};
within each unit independently x and y are either both CH or x is C and y is not present so that the respective unit corresponds to a spiro cyclic moiety of formula (SC) 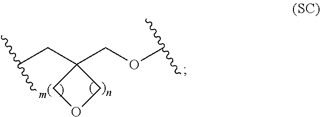
m is independently 0, 1, or 2;
n is independently 0, 1, or 2;
wherein within one unit when y is not present at least one of m or n is 1; p is an integer of 1 to 6;
R^{A} is a moiety of formula (A-1) or formula (A-2) 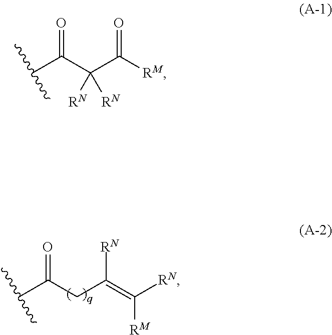
wherein
R^{N} is independently H, halogen, or C1-C3-alkyl;
R^{M} is C1-C6-alkyl or C1-C6-haloalkyl;
R⁸ is halogen, C1-C6-alkyl, or C1-C6-alkoxy;
t is O or an integer of 1 to 10000; and
q is O or an integer of 1 to 6,
and an additive (A) selected from the group consisting of catalyst (C2), reinforcement fibres, a filler, compounds comprising at least one α,β-unsaturated carbonyl group, epoxies, vinyl compounds, oxetanes, amines, isocyanates, and combinations thereof.

12. The composition according to claim 11, wherein one or more of the catalyst (C2) is selected from the group consisting of aliphatic mono-, di- and polyamines; aromatic mono-, di- and polyamines; carbocyclic mono-, di and polyamines; heterocyclic mono-, di- and polyamines; compounds containing a five- or six-membered nitrogen-containing heterocyclic ring; hydroxyamines; phosphines; phenols; and mixtures thereof, or
wherein the filler is selected from the group consisting of organic fillers; inorganic fillers; and mineral powder fillers; and mixtures thereof.

13. A process of manufacturing a 3D-printed object, a coating, an adhesive, a composite or construction material comprising the steps of
i) contacting a composition comprising a compound of formula (10) as defined in claim 11 with an additive (A) selected from the group consisting of catalyst (C2), reinforcement fibres, a filler, compounds comprising at least one α,β-unsaturated carbonyl group, epoxies, vinyl compounds, oxetanes, amines, isocyanates, and combinations thereof to provide a mixture (M); and
ii) curing said mixture (M).

14. A 3D-printed object, a coating, an adhesive, a composite or construction material obtainable by a process according to claim 13.

15. Use of a compound according to claim 1 in 3D-printing or for producing a coating, an adhesive, a composite or construction material.

16. The use according to claim 15 for producing a coating, wherein the coating comprises polyurea, polyurethane, and combinations thereof or wherein the coating comprises condensation products of epoxies and amines.

17. A method for fastening anchoring elements in boreholes, wherein a composition according to claim 11 is placed in the boreholes and the anchoring element is inserted therein.

18. A 3D printing resin composition comprising a compound of formula (10a) 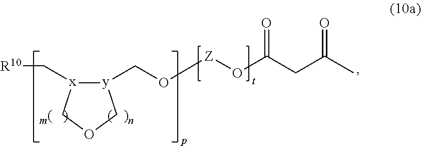
wherein
R¹⁰ is H, OH, C1-C10-alkyl, C1-C10-haloalkyl, C1-C10-alkoxy, C2-C10-alkenyl, C2-C10-alkynyl, C3-C10-cycloalkyl, C3-C10-cycloalkenyl, C3-C10-aryl, C3-C10-aryloxy, or OR^{A};
Z is within each unit independently C1-C3-alkylene, wherein each carbon atom of the aforementioned substituents may independently be unsubstituted or substituted with one or more same of different of R^{B};
within each unit independently x and y are either both CH or x is C and y is not present so that the respective unit corresponds to a spiro cyclic moiety of formula (SC) 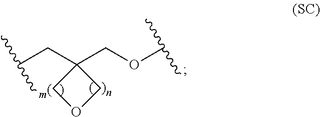
m is independently 0, 1, or 2;
n is independently 0, 1, or 2;
wherein within one unit when y is not present at least one of m or n is 1; p is an integer of 1 to 6;
R^{A} is a moiety of formula (A-1) or formula (A-2) 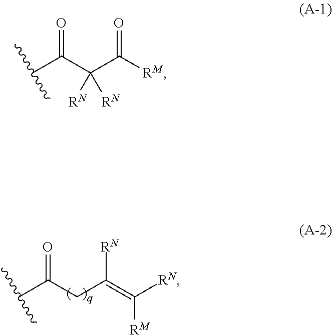
wherein
R^{N} is independently H, halogen, or C1-C3-alkyl;
R^{M} is C1-C6-alkyl or C1-C6-haloalkyl;
R⁸ is halogen, C1-C6-alkyl, or C1-C6-alkoxy;
t is O or an integer of 1 to 10000; and
q is O or an integer of 1 to 6.
